(19) United States
(12) Patent Application Publication
(10) Pub. No.: US 2004/0055022 A1
(43) Pub. Date: Mar. 18, 2004

(54) Compositions and methods for screening therapeutic agents

(76) Inventors: Nick Cheng, Coquitlam (CA); Liane Gagnier, Vancouver (CA); Wilfred A. Jefferies, South Surrey (CA)

Correspondence Address: TOWNSEND AND TOWNSEND AND CREW, LLP, TWO EMBARCADERO CENTER, EIGHTH FLOOR, SAN FRANCISCO, CA, 94111-3834, US

(21) Appl. No.: 10/181,937

(22) PCT Filed: Feb. 8, 2001
(86) PCT No.: PCT/CA01/00133

(51) Int. Cl. A61K 31/00; A01K 67/00

(52) U.S. Cl. 800/3; 800/12; 514/1

(57) ABSTRACT

Methods and models for transporting agents across the blood brain barrier, the preparation of antibodies and antisense oligonucleotides, the preparation of experimental systems to study murine p97, the isolation of substances that modulate murine p97 expression and/or activity as well as the use of the murine p97 nucleic acid sequences and proteins and modulators thereof in diagnostic and therapeutic applications are described.

### FIELD OF THE INVENTION

The invention relates to methods and experimental models for testing therapeutic agents and for screening for agents that modulate murine p97.

### BACKGROUND OF THE INVENTION

A major obstacle to the treatment of diseases of the brain, such as malignancy, Alzheimer's disease, Parkinson's disease, bacterial and viral infections, and deficiency diseases (e.g. Wernicke's disease and nutritional polyneuropathy) is the lack of an efficient and non-invasive means to deliver therapeutic agents across the blood brain barrier. Drug and solute transport into the brain from blood is restricted by the limited permeability of the brain capillary endothelial wall due to the endothelial tight junctions and the lack of aqueous pores in the endothelial cells (Pardridge, W. M. et al., J. Pharmacol. & Expt. Therapeut. 253: 884-891, 1990). Jefferies et al. (PCT International Publication No. WO 94/01463) discloses the use of a “shuttle” protein, p97, to transport therapeutic agents coupled to it across the blood brain barrier, the blood eye barrier and the blood placenta barrier.

Human p97 (hp97, alternatively known as melanotransferrin or human melanoma tumor-associated antigen) was one of the first cell surface markers to be associated with human skin cancer (Brown et al., J. Immunol., 127: 539-546, 1981; Hellstrom et al, Int. J. Cancer 31: 553-555, 1983). P97 belongs to a group of closely related iron binding proteins found in vertebrates (Rose, T. M. et al., Proc. Nat. Acad. Sci. U.S.A. 83: 1261, 1986). This family includes serum transferrin, lactoferrin and avian egg white ovotransferrin. P97 is a scialoglycoprotein and is encoded on chromosome 3 in humans (Plowman et al., Nature 303: 70-72, 1983. Human p97 and lactoferrin share a 40% sequence homology. However, p97 appears to be unique among the members of the transferrin family in that it has been shown to be connected to the cell membrane by a glycosyl-phosphatidylinositol (GPI) anchor (Alemany et al., J. Cell. Sci. 104: 1155-1162; Food et al., J.Biol. Chem 269: 3034-3040, 1994).

P97 is expressed on cultured normal cell types, including liver cells, intestinal epithelial cells, fetal cells, intestinal cells, umbilical chord, placenta and sweat gland ducts. More recently, p97 was shown to be expressed on normal capillary endothelial cells of human brain and reactive microglia of Alzheimer's disease patients (PCT International Publication No. WO 94/01463; Jefferies et al.). In addition, a soluble form of p97, which lacks the GPI anchor, has been found to be elevated in serum and other bodily fluids of Alzheimer's Disease patients (PCT International Publication No. WO 94/01463; PCT Application No. CA96/00587; Kennard et al., Nature Medicine 2: 1230-1235, 1996). It has also been demonstrated that p97 provides a novel route for cellular iron uptake which is independent of Tf and its receptor (U.S. Pat. No. 5,981,194 and Kennard 1995). The inventors have also demonstrated that p97 and TR express coincidentally in human brain capillary system, whereas Tf mainly localizes to glial cells, (U.S. Pat. No. 5,981,194 and Rothenberger 1996), which suggests that MTf may play a role in iron transport within the brain. In addition, p97 expressed on the brain endothelial cells is resistant to PI-PLC digestion suggesting that it is the soluble form of p97 bound to TR. Moreover reactive microglia specifically associated with amyloid plaque express p97 in Alzheimer disease brain (U.S. Pat. No. 5,981,194 and Jefferies 1996), and serum levels of the p97 as well as that of CSF levels significantly elevate in Alzheimer disease patients (U.S. Pat. No. 5,981,194 and Kennard 1996, supra). These data suggest p97 originating from reactive microglia appears in serum by crossing specific transport system existing at the blood brain barrier, and also implies the possibility of transcytosis from blood stream into the blood brain barrier when injected. A number of studies (summarized in Jefferies et al, Trends in Cell Biology 6: 229-228, 1996) suggest that p97 may play an active role in the transcytosis of iron across the blood brain barrier.

Human p97 has been cloned and expressed (U.S. Pat. Nos. 5,262,177, 5,141,742.) and is available for use in treatment protocols wherein therapeutic agents are bound or coupled to it. However, pre-clinical screening and in vivo testing of various therapeutic agents in association with p97 has been hampered by the lack of an inexpensive and convenient homologous test system. Although, for example, a heterologous test system using human p97 in a mouse model can be used, it would be useful to have a homologous test system which will reflect the homologous clinical situation, in which human p97 will be used to transport therapeutic substances across the blood brain barrier in humans. In order to provide more accurate information about the efficacy of specific p97-drug combinations, and to provide a rapid screening system for potential therapeutics, there is a need for a homologous animal model in which to test p97-coupled therapeutic agents.

In addition, in order to further elucidate the physiological role of p97 in vivo, it would be desirable to have a homologous mouse p97 in order to do p97 “knockout” or overexpression experiments in a mouse system.

### SUMMARY OF THE INVENTION

In one aspect, the invention provides isolated mouse p97 (hereinafter “mp97”) polypeptides having the amino acid sequence of SEQ.ID.NO.: 2, as well as polypeptides containing a portion of that amino acid sequence, and methods for their production. A preferred embodiment is a truncated mp97, that lacks a transmembrane portion, comprising amino acids 1-718 of SEQ.ID.NO.: 2.

In another aspect, the invention provides isolated polynucleotides encoding the mp97 protein having the nucleotide sequence of SEQ.ID.NO.: 1.

Another aspect of the invention provides methods for screening therapeutic compositions for their ability to cross the blood brain barrier and exert a therapeutic effect.

The invention also includes experimental models, including cells and animals, to identify modulators of p97 and to study its role in vivo.

### BRIEF DESCRIPTION OF THE DRAWINGS

The invention will now be described in relation to the drawings in which:

FIG. 1 is a schematic drawing showing of the use of circular RT-PCR to amplify the 5′ end of mp97 cDNA.

FIG. 2 is a schematic diagram of a mp97 cDNA.

FIG. 3 is a schematic diagram of the mp97 protein structure.

FIG. 4 is a schematic diagram comparing mp97 and hp97 protein structure.

FIG. 5 is a schematic diagram illustrating conserved structural features between the mouse and human p97 proteins.

### DETAILED DESCRIPTION OF THE INVENTION

The present invention is directed to mp97 polypeptides, polynucleotides encoding them, and their use in model systems for evaluating therapeutic agents and for identifying substances that modulate p97.

I. Murine p97 Polynucleotides

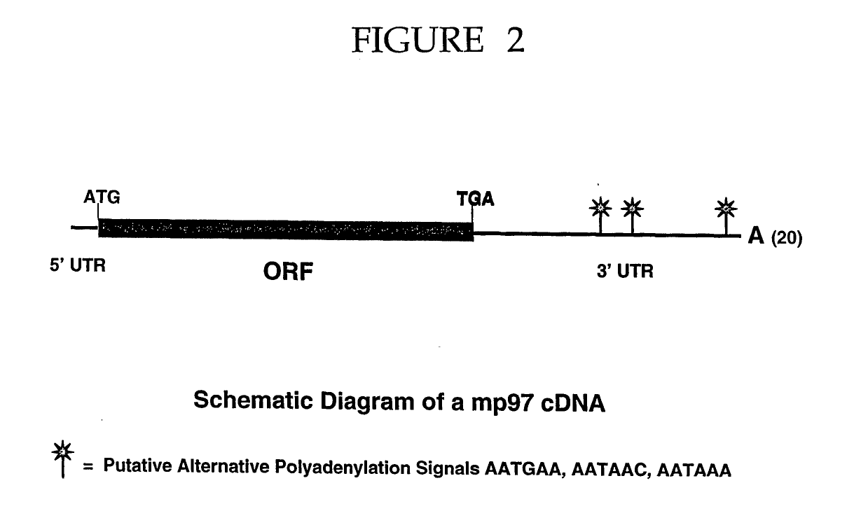

In one aspect, the invention provides polynucleotide sequences encoding mp97 polypeptides, including the p97 protein, which is presented in SEQ.ID.NO.: 1. An analysis of SEQ.ID.NO.: 1 revealed the following features, which are shown schematically in FIG. 2.:

1. 1-63: 5′ untranslated region (UTR);

2. 64-66: translational start codon ATG;

3. 64-2277: open reading frame (ORF) for the mouse p97mp97 protein;

4. 1063: a single nucleotide C deletion in EST2, wild-type in the 731 bp RT-PCR product;

5. 2278-2280: translational terminal codon TGA;

6. 2281-4068: 3′ UTR;

7. 3299-3304: putative alternative polyadenylation signal I, AATAAC;

8. 3544: alternative polyadenylation site I;

9. 3106-3111: putative polyadenylation signal II, AATGAA;

10. 3128: alternative polyadenylation site II;

11. 4028-4033: putative polyadenylation signal AATAAA for the EST2 transcript;

12. 4048: polyadenylation site for the EST2 transcript;

13. 4049-4068: polyadenylation tail (A=20); and

14. 491-1221: overlaps with the 731 bp RT-PCR product from the mouse melanoma cell line JB/MS that contains the wildtype sequence (without the single nucleotide C deletion at 1063).

The inventors isolated a DNA encoding mp97 cDNA as described in detail in Example 1 below. This involved using databases containing EST sequences to identify a 565 base pair fragment having 79% cDNA homology to a region in the 3′ region of the human p97 cDNA. (The database records do not disclose any polypeptide encoded by the 565 base pair EST, and do not indicate what the reading frame, if any, might be.) To extend the incomplete part of the cDNA, RACE PCR was performed on poly A+ RNA from a mouse melanoma cell line, JB/MS, using poly dT and internal primers designed from the putative mp97 cDNA. Alternatively the mp97 cDNA could be obtained by screening one or more cDNA libraries generated in a suitable host such as lambda gt 10 using poly A+ RNA from a p97 positive mouse cell line or tissue. Cell lines or tissues expressing mp97 can be identified by screening cytoplasmic RNA, preferably poly A+ RNA, for the ability to hybridize to human p97 cDNA. Clones which contain sequences encoding human p97 cDNA have been deposited with the American Type Culture Collection (ATCC) under deposit numbers CRL 8985 (PMTp97b) and CRL 9304 (pSVp97a). The clones containing mp97 cDNA are identified by their ability to hybridize under stringent conditions with labeled nucleic acid probes generated from the putative mp97 cDNA fragment, and/or the full length human p97 cDNA.

A preferred embodiment of the invention provides isolated DNA comprising a nucleotide sequence selected from the group consisting of nucleotides 64-2277 of SEQ.ID.NO.: 1, the mp97 coding region. DNAs fragments of SEQ.ID.NO.: 1 that code for portions of mp97 protein that are capable of acting as a shuttle to transport agents across the blood brain barrier are also included in the scope of the invention. Such DNA fragments can be identified by expressing the encoded polypeptide in a suitable system, labelling, and testing in an in vitro or in vivo model to determine whether it is capable of crossing the blood brain barrier. Methods for all of these steps are presented below. Other preferred embodiments and methods of production and use are discussed in more detail below. The mp97 polynucleotides or nucleic acids of the invention include cDNA, chemically synthesized DNA, DNA isolated by PCR, genomic DNA and combinations thereof. Genomic p97 DNA may be isolated from a genomic DNA library by hybridization to the mouse p97 cDNA disclosed herein using standard techniques. RNA transcribed from the mp97 DNA is also encompassed by the invention.

Accordingly, the present invention provides a substantially isolated nucleic acid sequence encoding a mp97 protein wherein the mp97 protein has at least 80% sequence identity with SEQ.ID.NO.: 1. Preferably, the nucleic acid sequence comprises:

(a) a nucleic acid sequence as shown in SEQ.ID.NO.: 1 wherein T can also be U;

(b) a nucleic acid sequence that is complimentary to a nucleic acid sequence of (a);

(c) a nucleic acid sequence that has substantial sequence homology to a nucleic acid sequence of (a) or (b);

(d) a nucleic acid sequence that is an analog of a nucleic acid sequence of (a), (b) or (c); or

(e) a nucleic acid sequence that hybridizes to a nucleic acid sequence of (a), (b), (c) or (d) under stringent hybridization conditions.

The term “sequence that has substantial sequence homology” means those nucleic acid sequences which have slight or inconsequential sequence variations from the sequences in (a) or (b), i.e., the sequences function in substantially the same manner. The variations may be attributable to local mutations or structural modifications. Nucleic acid sequences having substantial homology include nucleic acid sequences having at least 65%, more preferably at least 85%, and most preferably 90-95% identity with the nucleic acid sequences as shown in SEQ.ID.NO.: 1.

The term “sequence that hybridizes” means a nucleic. acid sequence that can hybridize to a sequence of (a), (b), (c) or (d) under stringent hybridization conditions. Appropriate “stringent hybridization conditions” which promote DNA hybridization are known to those skilled in the art, or may be found in Current Protocols in Molecular Biology, John Wiley & Sons, N.Y. (1989), 6.3.1-6.3.6. For example, the following may be employed: 6.0× sodium chloride/sodium citrate (SSC) at about 45° C., followed by a wash of 2.0×SSC at 50° C.; 0.2×SSC at 50° C. to 65° C.; or 2.0×SSC at 44° C. to 50° C. The stringency may be selected based on the conditions used in the wash step. For example, the salt concentration in the wash step can be selected from a high stringency of about 0.2×SSC at 50° C. In addition, the temperature in the wash step can be at high stringency conditions, at about 65° C.

The term “a nucleic acid sequence which is an analog” means a nucleic acid sequence which has been modified as compared to the sequence of (a), (b) or (c) wherein the modification does not alter the utility of the sequence as described herein. The modified sequence or analog may have improved properties over the sequence shown in (a), (b) or (c). One example of a modification to prepare an analog is to replace one of the naturally occurring bases (i.e. adenine, guanine, cytosine or thymidine) of the sequence shown in SEQ.ID.NO.: 1, with a modified base such as such as xanthine, hypoxanthine, 2-aminoadenine, 6-methyl, 2-propyl and other alkyl adenines, 5-halo uracil, 5-halo cytosine, 6-aza uracil, 6-aza cytosine and 6-aza thymine, pseudo uracil, 4-thiouracil, 8-halo adenine, 8-amninoadenine, 8-thiol adenine, 8-thiolalkyl adenines, 8-hydroxyl adenine and other 8-substituted adenines, 8-halo guanines, 8 amino guanine, 8-thiol guanine, 8-thiolalkyl guanines, 8-hydroxyl guanine and other 8-substituted guanines, other aza and deaza uracils, thymidines, cytosines, adenines, or guanines, 5-trifluoromethyl uracil and 5-trifluoro cytosine.

Another example of a modification is to include modified phosphorous or oxygen heteroatoms in the phosphate backbone, short chain alkyl or cycloalkyl intersugar linkages or short chain heteroatomic or heterocyclic intersugar linkages in the nucleic acid molecule shown in SEQ.ID.NO.: 1. For example, the nucleic acid sequences may contain phosphorothioates, phosphotriesters, methyl phosphonates, and phosphorodithioates.

A further example of an analog of a nucleic acid molecule of the invention is a peptide nucleic acid (PNA) wherein the deoxyribose (or ribose) phosphate backbone in the DNA (or RNA), is replaced with a polyamide backbone which is similar to that found in peptides (P. E. Nielsen, et al Science 1991, 254, 1497). PNA analogs have been shown to be resistant to degradation by enzymes and to have extended lives in vivo and in vitro. PNAs also bind stronger to a complimentary DNA sequence due to the lack of charge repulsion between the PNA strand and the DNA strand. Other nucleic acid analogs may contain nucleotides containing polymer backbones, cyclic backbones, or acyclic backbones. For example, the nucleotides may have morpholino backbone structures (U.S. Pat. No. 5,034,506). The analogs may also contain groups such as reporter groups, a group for improving the pharmacokinetic or pharmacodynamic properties of nucleic acid sequence.

II. Murine p97 Polypeptides

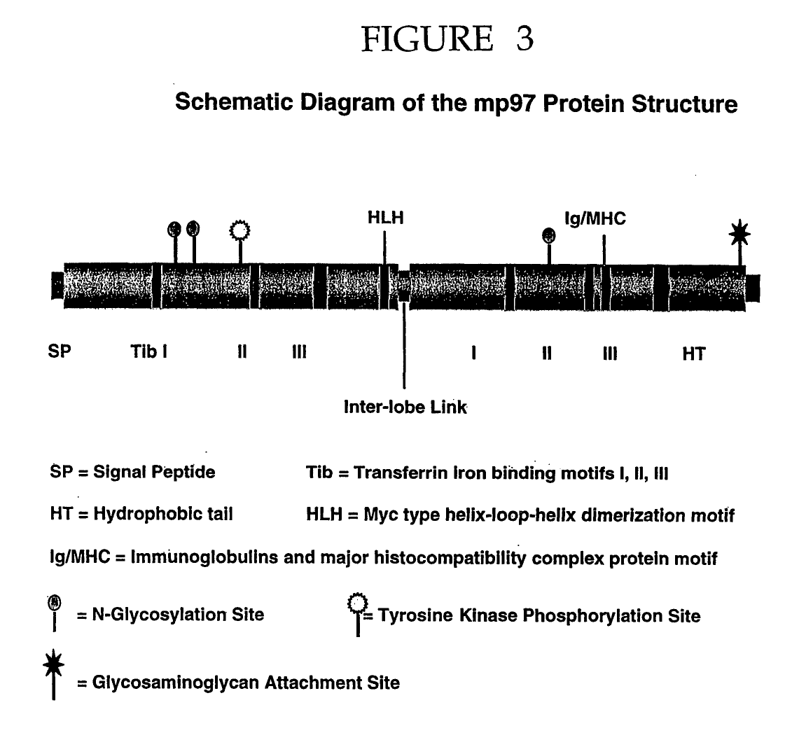

The amino acid sequence of mp97 protein encoded by the cDNA of SEQ.ID.NO.: 1 is presented in SEQ.ID.NO.: 2. The predicted mouse p97 protein from the cDNA sequence is composed of 738 a.a. with a molecular weight of 81,294 Da and a theoretical pI of 5.69. The first 19 amino acids at the N-terminal, MRLLSVTFWLLLSLRTVVC, is a signal peptide predicted by the method of Nielson, H. et al., Protein Engineering, 10, 1-6 1997. The most likely cleavage of the signal peptide lies between positions 19 and 20, VVC-VM, producing a mature protein with a molecular weight of 79,061 Da and a pI of 5.59. An analysis of the amino acid sequence revealed the following conserved sequences and potential functional motifs, which are shown schematically in FIG. 3:

I. Linear Structural Features:

1. 19 a.a. signal peptide: MRLLSVTFWLLLSLRTVVC (1-19);

2. N-terminal lobe (20-356);

3. 9 a.a. Iinterlobe domain (357-365);

4. C-terminal lobe (366-738718);

5. Hydrophobic tail: VPLLALLLLTLAAGLLPRVL (719-738); and

6. Conserved Regions between the N and C Lobes: (23-356, 366-705)

II. Other Potential Functional Motifs:

1. N-glycosylation sites:

NVTI (118-121)

NRTV (135-138)

NASC (515-518)

2. Transferrin iron binding motifs:

Motif I

| | | |
|---|---|---|
| N-Lobe | YYAVAVVRRN | (107-116) |
| C-Lobe | YFVVAVARRD | (451-460) |

Motif II

| | | |
|---|---|---|
| N-Lobe | YSGAFRCLAEGAGDVAF | (210-226) |
| C-Lobe | YSGAFRCVEHAGDVAF | (556-572) |

Motif III

| | | |
|---|---|---|
| N-Lobe | DFQLLCRDGSRADITEWRRCHLAKVPAHAVV | (252-282) |
| C-Lobe | -------DYELLCPNGARAEVDQFQACN | (598-628) |
| | LAQMPSHAVM | |

3. Tyrosine kinase phosphorylation site: KSPLERYY (201-208)

4. Myc-typepye helix-loop-helix dimerization motif: STLELVPIA (328-336)

5. Immunoglobulins and major histocompatibility complex proteins motif: FRCLVEH (560-566)

6. Glycosaminoglycan attachment site: SGAG (710-713)

7. Hydrophobic tail: VPLLALLLLTLAAGLLPRVL (719-738)

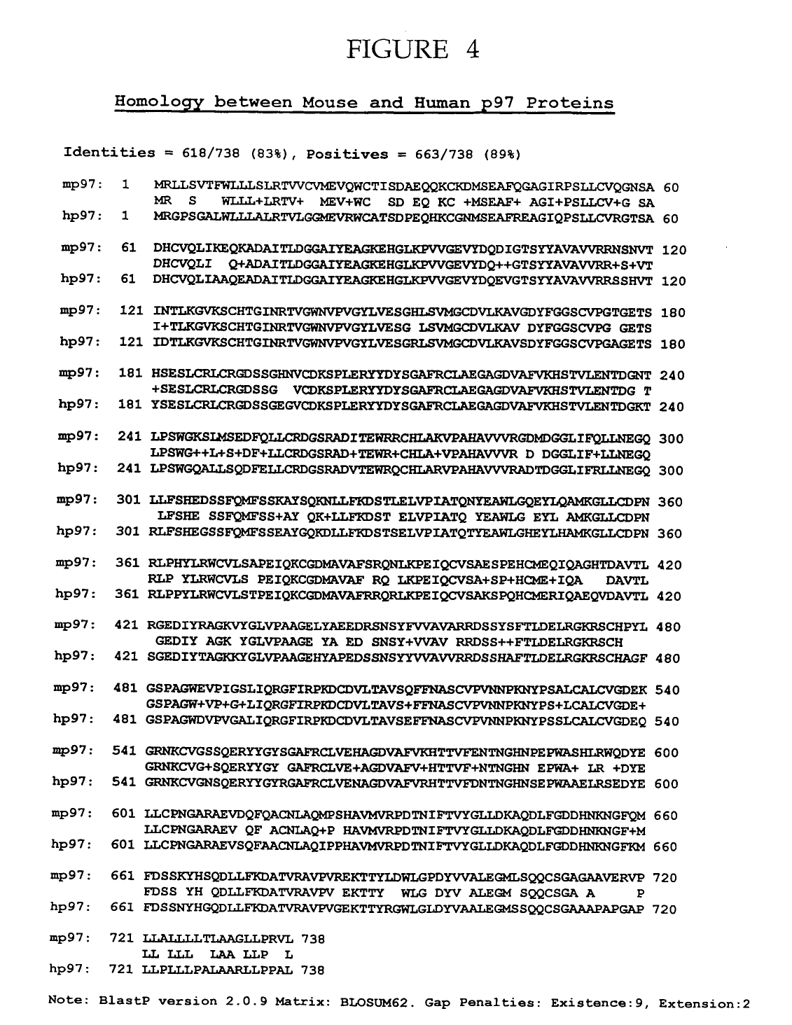

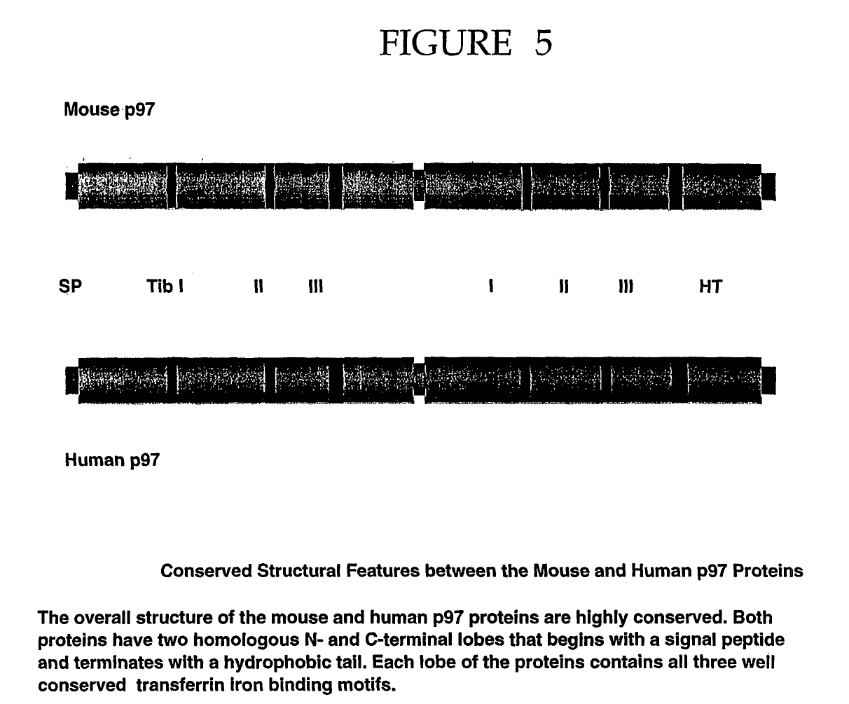

The p97 protein in human and mouse are highly conserved. They share 83% identity and 89% similarity in amino acid sequence. Their overall structure are similar (shown schematically in FIG. 4), both starting with a 19 a.a. signal peptide, then two conserved halves separated by a short interlobe domain, followed by a stretch of 20-27 hydrophobic amino acids at the C-terminal. The signal peptide and the hydrophobic tail are similar in sequence. More significantly, the three transferrin iron binding motifs and their locations within the protein are highly conserved, indicating the p97 protein in mouse and human plays a role in iron binding and transporting (see FIG. 5).

A mp97 polypeptide of the invention was obtained by expressing a vector containing cDNA encoding the polypeptide in a bacterial or mammalian cell culture expression system in Example 2. Methods for obtaining other p97 polypeptides which are within the scope of the invention are presented below.

Accordingly, the present invention provides a substantially isolated mp97 protein having at least 80% sequence identity with the amino acid sequence of SEQ.ID.NO.: 2.

Within the context of the present invention, p97 and derivatives thereof may include various structural forms of the primary protein which retain the ability to transport agents across the blood brain barrier. For example, a p97 protein may be in the form of acidic or basic salts, or in neutral form. In addition, individual amino acid residues may be modified by oxidation or reduction. Furthermore, various substitutions, deletions, or additions may be made to the amino acid or DNA nucleic acid sequences, the net effect of which is to retain biological activity or immunogenicity of mp97. Due to code degeneracy, for example, there may be considerable variation in nucleotide sequences encoding the same amino acid sequence.

Other derivatives of mp97 within the scope of this invention include conjugates of mp97 along with other molecules such as proteins or polypeptides as discussed below. This may be accomplished, for example, by the synthesis of N-terminal or C-terminal fusion or internally tagged proteins to facilitate purification or identification of mp97 (see U.S. Pat. No. 4,851,341, see also, Hopp et al., Bio/Technology 6:1204, 1988.) Fusion proteins may also be prepared for use in the compositions of the invention as discussed previously. Fusion proteins may be prepared by fusing, through recombinant techniques, the N-terminal or C-terminal of p97 or other portions thereof, and the sequence of a selected protein with a desired biological or therapeutic function. The resultant fusion proteins contain mp97 or a portion thereof fused to the selected protein. Examples of proteins which may be selected to prepare fusion proteins include lymphokines such as gamma interferon, tumor necrosis factor, IL-1, IL-2,IL-3, IL-4, IL-5, IL-6, IL-7, IL-8, IL-9, IL-10, IL-11, GM-CSF, CSF-1 and G-CSF. Particularly preferred molecules include nerve growth factor and the Fc portion of immunoglobulin molecules

Sequences which encode the above-described molecules may generally be obtained from a variety of sources, including for example, depositories which contain plasmids encoding sequences including the American Type Culture Collection (ATCC, Rockville Md.), and the British Biotechnology Limited (Cowley, Oxford England). Examples of such plasmids include BBG 12 (containing the GM-CSF gene coding for the mature protein of 127 amino acids), BBG 6 (which contains sequences encoding gamma interferon), ATCC No. 39656 (which contains sequences encoding TNF), ATCC No. 20663 (which contains sequences encoding alpha interferon,) ATCC Nos. 31902 and 39517 (which contains sequences encoding beta interferon), ATCC No. 67024 (which contains a sequence which encodes Interleukin-1b), ATCC Nos. 39405, 39452, 39516, 39626 and 39673 (which contains sequences encoding Interleukin-2), ATCC Nos. 59399, 59398, and 67326 (which contain sequences encoding Interleukin-3), ATCC Nos. 57592 (which contains sequences encoding Interleukin-4). ATCC Nos. 59394 and 59395 (which contain sequences encoding Interleukin-5), and ATCC No. 67153 (which contains sequences encoding Interleukin-6.

Expression of mp97 Proteins:

1. Full-length mp97 protein: To produce mp97 proteins, the mammalian expression vector pNUT was used. For full-length mp97, two constructs were made by cloning the EST2 cDNA into pNUT. Briefly, the cDNA was digested with XhoI completely and partially, and the cohesive ends were filled in by using Klenow. The 4.0 kb XhoI fragment of the entire cDNA from the partial digestion (with the internal XhoI site) and the 3.4 kb XhoI fragment with about 0.6 kb 3′ UTR deleted were gel purified. The pNUT plasmid was digested with SmaI followed by dephosphorylation by calf intestinal alkaline phosphatase. The linearized pNUT was gel purified and ligated with the mp97 XhoI fragments. Positive clones with the correct orientation were identified by diagnostic digestion using asymmetrically located restriction sites.

2. Secreted form of mp97: The C-terminal amino acid of the native secreted form of p97 has not yet been determined, either in human or in mouse. The 20 a.a. hydrophobic tail at the C-terminal is considered to be a signal required for addition of the GPI link. There are 13 a.a. from the hydrophobic tail to the region that are conserved with the N-lobe, and they have varied potential for being the GPI attachment site. These 13 a.a. are candidate sites for site-directed mutagenesis to truncate the C-terminal, thus creating a secreted form. By comparing the C-terminal end of the putative secreted and GPI linked forms of p97 in chicken, the last amino acid before the hydrophobic tail, Arg (coded by CGA), was chosen to convert into a translational stop (TGA). The mp97pNUT plasmid is used for U.S.E. mutagenesis (see before) and the internal XhoI site located in the 3′ UTR will be used as the unique selection site, converting into a SmaI site. The mutagenic and selection primers used are GGG GCC GCG GTC GAG TGA GTC CCC CTG G and CAT TTT GCC ATT GTT CTC CCG GGA ACC AGA AAA AGT TTT C respectively.

The U.S.E. mutagenesis, described above will introduce a premature stop codon immediately before the hydrophobic tail, creating a secreted form of mp97. Other forms of C-terminal deletion are carried out in similar fashion. N-terminal deletions are carried out using PCR based methods in combination with restriction digestion.

3. mp97 fusion proteins: To aid identification and purification of expressed proteins, full length p97 or truncated forms are fused with one or more of the following tags: His6-, flag-, or myc-tag. The fusion proteins are expressed in either a mammalian or bacterial system. For example, a His6 tag is attached at the C-terminal end of the secreted form of mp97. In brief, the pNUT plasmid that contains the mp97 EST2 is linearized by SacII, followed by dephosphorylation using calf intestinal alkline phophotase. Complementary oligos for His6-tag are synthesized with a SacII adaptor as following: 5′-G GTCGAG CGA CAT CAT CAT CAT CAT CAT TGA GC-3′, 5′-TCA ATG ATG ATG ATG ATG ATG TCG CTC GAC CGC-3′. The synthetic oligos are phosphorylated by T4 kinase, denatured, annealed, and then ligated with the prepared mp97 construct. Subsequently, the construct is transfected into mammalian cell lines for expression of a His tagged mp97 protein. The fusion protein is identified by using anti-His6 antibody and affinity purified using Nickle columns.

Mutations in nucleotide sequences constructed for expression of derivatives of p97 must preserve the reading frame phase of the coding sequences. Furthermore, the mutations will preferably not create complementary regions that could hybridize to produce secondary mRNA structures, such as loops or hairpins, which would adversely affect translation of the receptor mRNA.

Mutations may be introduced at particular loci by synthesizing oligonucleotides containing a mutant sequence, flanked by restriction sites enabling ligation to fragments of the native sequence. Following ligation, the resulting reconstructed sequence encodes a derivative having the desired amino acid insertion, substitution, or deletion.

Alternatively, as noted above oligonucleotide-directed site-specific mutagenesis procedures may be employed to provide an altered gene having particular codons altered according to the substitution, deletion, or insertion required. Deletion or truncation derivatives of p97 may also be constructed by utilizing convenient restriction endonuclease sites adjacent to the desired deletion. Subsequent to restriction, overhangs may be filled in, and the DNA religated. Exemplary methods of making the alterations set forth above are disclosed by Sambrook et al. (Molecular cloning A Laboratory Manual, 2d Ed., Cold Spring Harbor Laboratory Press, 1989).

As noted above, the present invention provides recombinant expression vectors which include either synthetic, or cDNA-derived DNA fragments encoding mp97 or derivatives thereof, which are operably linked to suitable transcriptional or translational regulatory elements. Suitable regulatory elements may be derived from a variety of sources, including bacterial, fungal, viral, mammalian, or insect genes. Selection of appropriate regulatory elements is dependent on the host cell chosen, and may be readily accomplished by one of ordinary skill in the art. Examples of regulatory elements include: a transcriptional promoter and enhancer or RNA polymerase binding sequence, a ribosomal binding sequence, including a translation initiation signal. Additionally, depending on the host cell chosen and the vector employed, other genetic elements, such as an origin of replication, additional DNA restriction sites, enhancers, sequences conferring inducibility of transcription, and selectable markers, may be incorporated into the expression vector.

DNA sequences encoding mp97 may be expressed by a wide variety of prokaryotic and eukaryotic host cells, including bacterial, mammalian, yeast or other fungi, viral, plant, or insect cells. Methods for transforming or transfecting such cells to express foreign DNA are well known in the art (see, e.g., Itakura et al., U.S. Pat. No. 4,704,362; Hinnen et al., PNAS USA 75:1929-1933, 1978; Murray et al., U.S. Pat. No. 4,801,542; Upshall et al., U.S. Pat. No. 4,935,349; Hagen et al., U.S. Pat. No. 4,784,950; Axel et al., U.S. Pat. No. 4,399,216; Goeddel et al., U.S. Pat. No. 4,766,075; and Sambrook et al. Molecular Cloning A Laboratory Manual, 2nd edition, Cold Spring Harbor Laboratory Press, 1989, all of which are incorporated herein by reference).

Bacterial host cells suitable for carrying out the present invention include E. coli, B. subtilis, Salmonella typhimuriurn, and various species within the genus' Pseudomonas, Streptomyces, and Staphylococcus, as well as many other bacterial species well known to one of ordinary skill in the art. Representative examples of bacterial host cells include DH5a (Stratagene, Lajolla, Calif.), JM109 ATCC No. 53323, HB101 ATCC No. 33694, and MN294.

Bacterial expression vectors preferably comprise a promoter which functions in the host cell, one or more selectable phenotypic markers, and a bacterial origin of replication. Representative promoters include the b-lactamase (penicillinase) and lactose promoter system (see Chang et al., Nature 275:615, 1978), the trp promoter (Nichols and Yanofsky, Meth in Enzymology 101:155, 1983) and the tac promoter (Russell et al., Gene 20: 231, 1982). Representative selectable markers include various antibiotic resistance markers such as the kanamycin or ampicillin resistance genes. Many plasmids suitable for transforming host cells are well known in the art, including among others, pBR322 (see Bolivar et al., Gene 2:9S, 1977), the pUC plasmids pUC18, pUC19, pUC118, pUC119 (see Messing, Meth in Enzymology 101:20-77, 1983 and Vieira and Messing, Gene 19:259-268, 1982), and pNH8A, pNH16a, pNH18a, and Bluescript M13 (Stratagene, La Jolla, Calif.).

Yeast and fungi host cells suitable for carrying out the present invention include, among others Saccharomyces cerevisiae, the genera Pichia or Kluyveromyces and various species of the genus Aspergillus. Suitable expression vectors for yeast and fungi include, among others, YCp50 (ATCC No. 37419) for yeast, and the amdS cloning vector pV3 (Turnbull, Bio/Technology 7:169, 1989). Protocols for the transformation of yeast are also well known to those of ordinary skill in the art. For example, transformation may be readily accomplished either by preparation of spheroplasts of yeast with DNA (see Hinnen et al., PNAS USA 75:1929, 1978) or by treatment with alkaline salts such as LiCl (see Itoh et al., J. Bacteriology 153:163, 1983). Transformation of fungi may also be carried out using polyethylene glycol as described by Cullen et al. (Bio/Technology 5:369, 1987).

Mammalian cells suitable for carrying out the present invention include, among others: COS (e.g., ATCC No. CRL 1650 or 1651), BHK (e.g., ATCC No. CRL 6281), CHO (ATCC No. CCL 61), HeLa (e.g., ATCC No. CCL 2), 293 (ATCC No. 1573) and NS-1 cells. Suitable expression vectors for directing expression in mammalian cells generally include a promoter, as well as other transcriptional and translational control sequences. Common promoters include SV40, MMTV, metallothionein-1, adenovirus Ela, CMV, immediate early, immunoglobulin heavy chain promoter and enhancer, and RSV-LTR. Protocols for the transfection of mammalian cells are well known to those of ordinary skill in the art. Representative methods include calcium phosphate mediated electroporation, retroviral, and protoplast fusion-mediated transfection (see Sambrook et al., supra).

Given the teachings provided herein, promoters, terminators, and methods for introducing expression vectors of an appropriate type into plant, avian, and insect cells may also be readily accomplished. For example, within one embodiment, mp97 or derivatives thereof may be expressed from plant cells (see Sinkar et al., J. Biosci (Bangalore) 11:47-58, 1987, which reviews the use of Agrobacterium rhizogenes vectors; see also Zambryski et al., Genetic Engineering, Principles and Methods, Hollaender and Setlow (eds.), Vol. VI, pp. 253-278, Plenum Press, New York, 1984, which describes the use of expression vectors for plant cells, including, among others, pAS2022, pAS2023, and pAS2034).

Within a particularly preferred embodiment of the invention, mp97 is expressed from baculoviruses, (see Example 2 below) (see also Luckow and Summers, Bio/Technology 6:47, 1988; Atkinson et al., Petic. Sci 28:215-224, 1990). Use of baculoviruses such as AcMNPV is particularly preferred due to the expression of GPI-cleaved forms of mp97 from the host insect cells.

mp97 may be prepared by culturing the host/vector systems described above, in order to express the recombinant mp97. Recombinantly produced mp97 may be further purified as described in more detail below.

In another example, mp97 may be isolated from cells which express mp97. The present inventors have developed methods for preparing a cleaved form of mp97 comprising the step of incubating a cell which expresses mp97 on its surface with an enzyme that cleaves phospholipid anchors, if the mp97 protein, like the human mp97 is anchored to the cell surface by a glycosyl-phosphatidylinositol (GPI) anchor. Various enzymes display a specificity toward GPI linkages, and thus may be utilized within the context of the present invention to cleave the GPI anchor. Representative examples include bacterial phosphatidyl inositol-phospholipase Cs (PI-PLCs) (see Ikezawa et al., Methods Enzymol. 71:731-741, 1981; Taguchi et al., Arch. Biochem. Biophys. 186:196-201, 1978; Low, Methods Enzymol. 71:741-746, 1981), eukaryotic GPI-PLCs (see Ferguson et al., J. Biol. Chem. 260:4963-68, 1985; Bulow et al., FEBS Lett. 187:105-110, 1985), and eukaryotic phospholipase Ds (GPI-PLD2 or “PLD”) (see Malik et al., Biochem. J. 240:519-527, 1986) (see generally, Ferguson and Williams, “Cell-Surface Anchoring of Proteins via Glycosyl-Phosphatidylinositol Structures”, Ann. Rev. Biochem. 57:285-320,1988).

A particularly preferred GPI enzyme is phospholipase C (PI-PLC) which may be obtained either from bacterial sources (see Low, “Phospholipase Purification and Quantification” The Practical Approach Series: Cumulative Methods Index, Rickwood and Hames, eds. IRC Press, Oxford, N.Y., N.Y., 1991; Kupe et al., Eur. J. Biochem. 185:151-155, 1989; Volwerk et al., J. Cell. Biochem. 39:315-325, 1989) or from recombinant sources (Koke et al., Protein Expression and Purification 2:51-58, 1991; and Henner et al., Nuc. Acids Res. 16:10383, 1986).

mp97 may be cleaved from the surface of a variety of cells which are found to express it as well as cells which have been infected or transfected with a vector which expresses mp97 (see below). If desired, the cleaved (solubilized) mp97 may then be purified utilizing techniques which are also described in more detail below, including affinity chromatography.

The soluble form of mp97 may be prepared by culturing cells which contain the soluble mp97 through the log phase of the cell's growth and collecting the supernatant. Preferably, the supernatant is collected prior to the time the cells reach confluency. Soluble mp97 may then be purified as described below, in order to yield isolated soluble mp97. Methods for purifying the soluble mp97 can be selected based on the hydrophilic property of the soluble mp97. For example, the soluble mp97 may be readily obtained by Triton X-114. Phase Separation.

In another example, mp97 may be isolated from CHO cells genetically engineered to express the GPI-anchored mp97 were grown in culture. The GPI-anchored protein may be harvested by a brief incubation with an enzyme capable of cleaving the GPI anchor, such enzymes are known in the art (Ferguson, M. J., Ann. Rev. Biochem. 57:285-320, 1988) and representative examples are described above. Preferably PI-PLC or GPI-PLC are used in the method of the invention. The cleaved soluble protein may be recovered from the medium and the cells returned to growth medium for further expression of the protein. Cycles of growth and harvest may be repeated until sufficient quantities of the protein are obtained.

In a preferred embodiment, CHO cells may be grown in spinner cultures on porous microcarriers such as Cultispher-GH porous microcarriers, solid microcarriers such as Cytodex-1, or spheroids.

Purification of Mouse p97

mp97 and derivatives thereof, as well as soluble mp97, may be readily purified given the teaching provided in Example 2 and elsewhere herein. Generally, mp97 may be purified either from supernatants containing solubilized mp97, or from cultured host/vector systems as described above. A variety of purification steps, used either alone or in combination may be utilized to purify mp97. For example, supernatants obtained by solubilizing mp97, or from host/vector cultures as described above, may be readily concentrated using commercially available protein concentration filters, for example, an Amicon or Millipore Pellicon ultrafiltration unit, or by “salting out” the protein followed by dialysis. In addition to concentration, supernatants (or concentrates) may be applied to an affinity purification matrix such as an anti-mp97 antibody which is bound to a suitable support. Alternatively, an anion exchange resin may be employed, for example, a matrix or substrate having pendant diethylaminoethyl (DEAE) groups. Representative matrices include acrylamide, agarose, dextran, cellulose or other types commonly employed in protein purification. Similarly, cation exchangers may be employed which utilize various insoluble matrices such as sulfopropyl or carboxymethyl groups.

Finally, one or more reversed-phase high performance liquid chromatography (RP-HPLC) steps employing hydrophobic RP-HPLC media, e.g, silica gel having pendant methyl or other alipathic groups, can be employed to further purify a glucagon receptor composition.

Within the context of the present invention, “isolated” or “purified”, as used to define the purity of mp97, means that the protein is substantially free of other proteins of natural or endogenous origin, and contains less than about 1% by mass of protein contaminants due to the residual of production processes. mp97 may be considered “isolated” if it is detectable as a single protein band upon SDSPAGE, followed by staining with Coomasie Blue.

III. Uses

The present invention includes all uses of the murine p97 nucleic acid molecules and murine p97 proteins of the invention including, but not limited to, methods and models for transporting agents across the blood brain barrier, the preparation of antibodies and antisense oligonucleotides, the preparation of experimental systems to study murine p97, the isolation of substances that modulate murine p97 expression and/or activity as well as the use of the murine p97 nucleic acid sequences and proteins and modulators thereof in diagnostic and therapeutic applications. Some of the uses are further described below.

(a) Compositions, Methods and Models for Transporting Therapeutic Agents Across the Blood Brain Barrier

The present invention provides a composition for transporting an agent across the blood brain barrier comprising (a) mp97 or a substance capable of binding mp97 in association with (b) the agent. In particular, the composition may contain mp97 conjugated to the agent; a mp97 fusion protein comprising mp97 or a portion thereof fused to the agent; a substance capable of binding to mp97, e.g. iron, or a substance capable of binding to mp97, e.g. anti-mp97 antibody, conjugated to the agent. Such compositions are herein referred to as “mp97-agent complexes”, or “mp97-therapeutic agent complexes.”

Accordingly, the present invention provides a method for assessing the ability of an agent to cross the blood brain barrier comprising (1) administering an effective amount of (a) the agent associated with- murine p97 or (b) the agent associated with a compound that binds murine p97 and (2) testing the levels of the agent in the nervous system.

The invention also provides a method to assess the ability of a therapeutic agent to treat a neurological condition, comprising (1) administering to a mouse an effective amount of (a) the agent associated with murine p97 or (b) the agent associated with a compound that binds murine p97 and (2) monitoring the result of administration wherein an improvement in the neurological condition indicates that the agent has therapeutic effect. Control mice, which received the carrier, but not the complex can be included in the monitoring. In some embodiments, the agent is labelled (for example, with 125I), so that the monitoring could include localizing the agent in the mouse following administration. Methods for labeling are described below. In other embodiments, the monitoring involves performing an assay for a desired pharmacological effect, or a desired behavioral effect. For example, after administration of a mp97-cytotoxic chemotherapeutic agent to a mouse having malignant brain metastases, the monitoring step would entail quantitating the size and/or number of metastases compared to control animals that did not receive the complex. In another embodiment, in which the complex comprised mp97 and an enzyme, the monitoring step would involve performing an assay for the enzyme on brain tissue. Such enzyme assays are well known in the art.

In a preferred embodiment, the mice to which the complex is administered have genetic defects leading to lysosomal enzyme deficiencies. It is well known in the art that deficient enzymes, if supplied intravenously, do not cross the blood barrier, and hence have no therapeutic effect. The mp97-enzyme complex is injected into the mice, and the mice are monitored for restoration of normal cell metabolism in the brain cells. The recovery can be assessed by microscopic analysis of brain tissues, since large vesicles appear in the brain cells of animals deficient in lysosomal enzymes. The disappearance of the large vesicles is indicative of the restoration of a normal phenotype.

In another preferred embodiment of the method of the invention, the mouse to which the mp97-agent complex is administered to an“Alzheimer's Disease (AD) prone mouse.” Hsiao et al. (Science 274: 99-102, 1996) have developed a transgenic mouse model for AD is available which shows similarities to the pathology of human AD in that the animals develop senile plaques, diffuse plaques, and possibly neurofibrial tangles. Most importantly, these animals develop clear memory defects. mp97-therapeutic agent complexes which are potential therapeutics for AD can be tested by administering them to the AD prone mice. The levels of mp97 in the serum or other bodily fluids of AD prone mice can be monitored before and after treatment. The assay for mp97 is essentially as described for human p97 in PCT Application No. CA96/00587 and Kennard et al., Nature Medicine 2: 1230-1235, 1996, which are incorporated herein by reference in their entirety, except that antibodies are against mp97 as detailed herein. A decrease the level of mp97 in the mouse serum would be one of the indicia of a successful therapeutic agent.

The method of the invention can also be used to refine a p97 polypeptide which is optimal for the delivery of any particular therapeutic agent or class of agents. For example, a therapeutic agent could be coupled to a full length secreted p97 protein, as well as various fragments or derivatives of p97 as described herein. The monitoring step would reveal the most suitable p97 polypeptide for delivery of that therapeutic agent or class of agents.

Various mp97-agent complexes of the invention may also be tested for their ability to cross the blood brain barrier and provide the desired pharmacological effect using in vitro models of the blood brain barrier. Examples of in vitro models include capillary endothelial cell lines, which in culture form an endothelial monolayer with high resistance to drug and solute transport (Pardridge, W. M. et al., J. Pharmacol. & Expt. Therapeut. 253:884-891, 1990).

Any route of administration of a mp97-agent complex to an intact mouse (or other animal) which dilutes the composition into the blood stream could be used. Preferably, the composition is administered peripherally, most preferably intravenously or by cardiac catheter. Dosages to be administered will depend on individual needs, on the desired effect and on the chosen route of administration.

Compositions of the invention may also be administered encapsulated in or attached to viral envelopes or vesicles or incorporated into cells. Vesicles are micellular particles which are usually spherical and which are frequently lipid. Liposomes are vesicles formed from a bilayered membrane. Suitable vesicles include unilamellar vesicles and multilamellar lipid vesicles or liposomes, which may be made from a wide range of lipid or phospholipid compounds, such as phosphatidylcholine, phosphatidic acid, phosphatidylserine, phosphatidylethanolamine, sphingomyelin, glycolipids, gangliosides etc. using known techniques, such as those described in U.S. Pat. No. 4,394,448. Such vesicles or liposomes may be used to administer compounds intracellularly and to deliver compounds across the blood brain barrier. Controlled release of the therapeutic agent may also be achieved by using encapsulation (U.S. Pat. No. 5,186,941).

The present invention also contemplates that compositions of the invention may be delivered across the blood eye and blood placenta barrier. Delivery across the blood placenta barrier is expected to have useful applications in gene therapy for providing recombinant DNA molecules to the foetus. In gene therapy, a functional gene may be introduced into a foetus in need to correct a genetic defect. The transfer of a recombinant DNA molecule into a mammalian foetus may be used, for example in gene therapy to correct an inherited or acquired disorder through the synthesis of missing or defective gene products in vivo. The recombinant DNA molecule may be incorporated into the above-noted vesicles, liposomes or viral envelopes. It is also contemplated that p97 and the delivery compositions of the invention may be useful for delivering therapeutic agents and pharmaceuticals, (e.g. antibiotics) across the blood placenta barrier as well as to other organs including liver. The compositions may also be used to test cancer therapies such as therapies for melanoma which expresses p97.

mp97 which may be used in the compositions of the invention include soluble mp97, cleaved mp97, and derivatives and portions thereof. Portions or peptides of mp97 may be used that contain a sufficient portion of mp97 to enable it to be transported across the blood brain barrier. Methods of preparing mp97 or portions thereof are described in detail herein. Antibodies to mp97 which may be used in the composition are also described in detail below.

Agents which may be used in the methods and compositions of the invention may be those known for the treatment of a neurological condition or suspected of having activity against a neurological condition. The term “neurological condition” as used herein means any condition affecting the nervous system including, but not limited to, cancers, neurodegenerative diseases (Alzheimer's disease, Parkinson's disease, Huntington's disease), demyelinating diseases (e.g. multiple sclerosis), amyotrophic lateral sclerosis, bacterial and viral infections, deficiency diseases (e.g. Wernicke's Disease and nutritional polyneuropathy), epilepsy, psychosis, pain and neurological disorders.

Accordingly, agents which may be used in the compositions of the invention include chemotherapeutics, antibiotics, cholinergic agonists, anticholinesterase agents, adrenergic receptor antagonists, drugs acting on the central nervous system and peripheral nervous system, neurotransmitters and neuropeptide hormones, sedatives, antipsychotic compounds and any other drug that acts on the nervous system.

In one embodiment, the composition is used to deliver an agent to the brain in the treatment of Alzheimer's disease. Possible therapeutic agents which can be used in the compositions for the treatment of Alzheimer's disease include, but are not limited to, iron sequestering compounds, such as iron chelators, and anti-inflammatory drugs. Proteins such as growth factors, including nerve growth factor, brain-derived neurotrophic factor, and lymphokines including gamma interferon, tumor necrosis factor, the interleukins, GM-CSF, CSF-1, and G-CSF are also contemplated as therapeutic agents for use in the delivery compositions of the invention. Cholinergic neurons of the basal forebrain, which degenerate in Alzheimer's disease, are known to depend on nerve growth factor for their survival. Nerve growth factor has also been shown to rescue degenerating cholinergic neurons in the forebrain (Hefti, F. J. Neurosci 6:2155, 1986).

Conjugates

Conjugates of mp97 or a substance that binds mp97 and the agent may be prepared using techniques known in the art. There are numerous approaches for the conjugation or chemical crosslinking of proteins and one skilled in the art can determine which method is appropriate for the therapeutic agent to be conjugated. The method employed must be capable of joining the agent with mp97 or a substance which binds mp97 without interfering with the ability of mp97 to bind to it's receptor and without significantly altering the activity of the therapeutic agent. If the therapeutic agent is a protein or a peptide, there are several hundred crosslinkers available in order to conjugate the agent with the mp97 or a substance which binds mp97. (See for example “Chemistry of Protein Conjugation and Crosslinking”. 1991, Shans Wong, CRC Press, Ann Arbor). The crosslinker is generally chosen based on the reactive functional groups available or inserted on the therapeutic agent. In addition, if there are no reactive groups a photoactivatible crosslinker can be used. In certain instances, it may be desirable to include a spacer between the mp97 or substance which binds mp97 and the therapeutic agent. In one example, mp97 or an antibody thereto and protein therapeutic agents may be conjugated by the introduction of a sulfhydryl group on the mp97 or antibody and the introduction of a cross-linker containing a reactive thiol group on to the protein agent through carboxyl groups (Wawizynczak, E. J. and Thorpe, P. E. in Immunoconjugates: Antibody Conjugates in Radioimaging and Therapy of Cancer, C. W. Vogel (Ed.) Oxford University Press, 1987, pp. 28-55.; and Blair, A. H. and T. I. Ghose, J. Immunol. Methods 59:129 ,1983).

mp97 can be crosslinked to peptides or polypeptides using SATA and a hetero-bifunctional cross-linker, Sulfo-SMCC, both available from Pierce. Activation of mp97 with the NHS half of sulfo-SMCC (reacts with primary amines) and other proteins with the NHS half of SATA (which introduces protected sulfhydryl groups on primary amines). After deprotection to a —SH group, the maleimide half of the sulfo-SMCC from mp97 can react with the free —SH group of the other polypeptide to be cross-linked. Alternatively, the polypeptide or mp97 can be activated by periodate, and then reacted with the other compound. Another alternative is the Streptavidin-Biotin method.

Fusion Proteins

Fusion proteins of mp97 or a substance that binds mp97 and a protein or peptide therapeutic agent may be prepared using techniques known in the art. In such a case, a DNA molecule encoding mp97 or a portion thereof is linked to a DNA molecule encoding the therapeutic agent. The chimeric DNA construct, along with suitable regulatory elements can be cloned into an expression vector and expressed in a suitable host. Methods for preparing fusion proteins are described in greater detail above.

Preparations of Antibodies to mp97

Antibodies to a mp97 polypeptide were raised as described in Example 3. Generally, mp97 or derivatives thereof, soluble mp97, or cells which contain mp97 on their surface (including cells transfected with mp97 DNA) may be utilized to prepare antibodies. Within the context of the present invention, antibodies are understood to include monoclonal antibodies, polyclonal antibodies, antibody fragments (e.g., Fab, and F(ab′)2 and recombinantly produced binding partners. Antibodies are understood to be reactive against mp97 if it binds with a Ka of greater than or equal to 10−7 M. As will be appreciated by one of ordinary skill in the art, antibodies may be developed which not only bind to a ligand such as mp97, but which also block the biological activity of the ligand (e.g, by blocking the binding of iron or transferrin receptor to mp97).

Polyclonal antibodies may be readily generated by one of ordinary skill in the art from a variety of warm-blooded animals such as horses, cows, various fowl, rabbits, or rats. Briefly, mp97 is utilized to immunize the animal through intraperitoneal, intramuscular, intraocular, or subcutaneous injections, an adjuvant such as Freund's complete or incomplete adjuvant. Following several booster immunizations, samples of serum are collected and tested for reactivity to mp97. Particularly preferred polyclonal antisera will give a signal on one of these assays that is at least three times greater than background. Once the titer of the animal has reached a plateau in terms of its reactivity to mp97, larger quantities of antisera may be readily obtained either by weekly bleedings, or by exsanguinating the animal.

Monoclonal antibodies may also be readily generated using conventional techniques (see U.S. Pat. Nos. RE 32,011, 4,902,614, 4,543,439, and 4,411,993 which are incorporated herein by reference; see also Monoclonal Antibodies, Hybridomas: A New Dimension in Biological Analyses, Plenum Press, Kennett, McKearn, and Bechtol (eds.), 1980, and Antibodies: A Laboratory Manual, Harlow and Lane (eds.), Cold Spring Harbor Laboratory Press, 1988, which are also incorporated herein by reference).

Briefly, within one embodiment a subject animal such as a rabbit is injected with mp97. The mp97 may be admixed with an adjuvant such as Freund's complete or incomplete adjuvant in order to increase the resultant immune response. Between one and three weeks after the initial immunization the animal may be reimmunized with another booster immunization, and tested for reactivity to mp97 using assays described above. Once the animal has plateaued in its reactivity to mp97, it is sacrificed, and organs which contain large numbers of B cells such as the spleen and lymph nodes are harvested.

Cells which are obtained from the immunized animal may be immortalized by transfection with a virus such as the Epstein bar virus (EBV) (see Glasky and Reading, Hybridoma 8(4):377-389, 1989). Alternatively, within a preferred embodiment, the harvested spleen and/or lymph node cell suspensions are fused with a suitable myeloma cell in order to create a “hybridoma” which secretes monoclonal antibody. Suitable myeloma lines include, for example, NS-1 (ATCC No. TIB 18), and P3×63—Ag 8.653 (ATCC No. CRL 1580).

Following the fusion, the cells may be placed into culture plates containing a suitable medium, such as RPMI 1640, or DMEM (Dulbecco's Modified Eagles Medium) (JRH Biosciences, Lenexa, Kansas), as well as additional ingredients, such as Fetal Bovine Serum (FBS, ie., from Hyclone, Logan, Utah, or JRH Biosciences). Additionally, the medium should contain a reagent which selectively allows for the growth of fused spleen and myeloma cells such as HAT (hypoxanthine, aminopterin, and thymidine) (Sigma Chemical Co., St. Louis, Mo.). After about seven days, the resulting fused cells or hybridomas may be screened in order to determine the presence of antibodies which are reactive against mp97. A wide variety of assays may be utilized to determine the presence of antibodies which are reactive against mp97, including for example Countercurrent Immuno-Electrophoresis, Radioimmunoassays, Radioimmunoprecipitations, Enzyme-Linked Immuno-Sorbent Assays (ELISA), Dot Blot assays, inhibition or Competition Assays, and sandwich assays (see U.S. Pat. Nos. 4,376,110 and 4,186,530; see also Antibodies: A Laboratory Manual, Harlow and Lane (eds.), Cold Spring Harbor Laboratory Press, 1988). Following several clonal dilutions and reassays, a hybridoma producing antibodies reactive against mp97 may be isolated.

Other techniques may also be utilized to construct monoclonal antibodies (see William D. Huse et al., “Generation of a Large Combinational Library of the Immunoglobulin Repertoire in Phage Lambda”, Science 246:1275-1281, December 1989; see also L. Sastry et al., “Cloning of the Immunological Repertoire in Escherichia coli for Generation of Monoclonal Catalytic Antibodies: Construction of a Heavy Chain Variable Region-Specific cDNA Library”, Proc Natl. Acad. Sci USA 86:5728-5732, August 1989; see also Michelle Alting-Mees et al., “Monoclonal Antibody Expression Libraries: A Rapid Alternative to Hybridomas”, Strategies in Molecular Biology 3:1-9, January 1990; these references describe a commercial system available from Stratacyte, La Jolla, Calif., which enables the production of antibodies through recombinant techniques). Briefly, mRNA is isolated from a B cell population, and utilized to create heavy and light chain immunoglobulin cDNA expression libraries in the 1ImmunoZap(H) and 1ImmunoZap(L) vectors. These vectors may be screened individually or co-expressed to form Fab fragments or antibodies (see Huse et al. supra; see also Sastry et al., supra). Positive plaques may subsequently be converted to a non-lytic plasmid which allows high level expression of monoclonal antibody fragments from E. coli.

Similarly, binding partners may also be constructed utilizing recombinant DNA techniques to incorporate the variable regions of a gene which encodes a specifically binding antibody. Within one embodiment, the genes which encode the variable region from a hybridoma producing a monoclonal antibody of interest are amplified using nucleotide primers for the variable region. These primers may be synthesized by one of ordinary skill in the art, or may be purchased from commercially available sources. Stratacyte (La Jolla, Calif.) sells primers for mouse and human variable regions including, among others, primers for VHa, VHb, VHc, VHd, CH1, VL and CL regions. These primers may be utilized to amplify heavy or light chain variable regions, which may then be inserted into vectors such as ImmunoZAP™ H or ImmunoZAP™ L (Stratacyte), respectively. These vectors may then be introduced into E. coli for expression. Utilizing these techniques, large amounts of a single-chain protein containing a fusion of the VH and VL domains may be produced (See Bird et al., Science 242:423426, 1988). In addition, such techniques may be utilized to change a “murine” antibody to a “human” antibody, without altering the binding specificity of the antibody.

Once suitable antibodies or binding partners have been obtained, they may be isolated or purified by many techniques well known to those of ordinary skill in the art (see Antibodies: A Laboratory Manual, Harlow and Lane (eds.), Cold Spring Harbor Laboratory Press, 1988). Suitable techniques include peptide or protein affinity columns, HPLC or RP-HPLC, purification on protein A or protein G columns, or any combination of these techniques.

Labelling of mp97

mp97, soluble mp97, cleaved mp97, GPI-anchored mp97, and derivatives thereof, and antibodies which are described above may be labelled with a variety of molecules, including for example, fluorescent molecules, toxins, substances having therapeutic activity i.e. therapeutic agents, luminescent molecules, enzymes, and radionuclides. Representative examples of fluorescent molecules include fluorescien, phycoerythrin, rodamine, Texas red and luciferase. Representative examples of toxins include ricin, abrin diptheria toxin, cholera toxin, gelonin, pokeweed antiviral protein, tritin, Shigella toxin, and Pseudomonas exotoxin A. Representative examples of radionuclides include Cu-64, Ga-67, Ga-68, Zr-89, Ru-97, Tc-99m, Rh-105, Pd-109, In-111, I-123, I-125, I-131, Re-186, Re-188, Au-198, Au-199, Pb-203, At-211, Pb-212 and Bi-212. Examples of suitable enzymes include horseradish peroxidase, biotin, alkaline phosphatase, β-galactosidase, or acetylcholinesterase; and an example of a luminescent material includes luminol. In addition, the mp97 or antibodies described above may also be labelled or conjugated to one partner of a ligand binding pair. Representative examples include avidin-biotin, and riboflavin-riboflavin binding protein.

Methods for conjugating or labelling the mp97 or antibodies discussed above with the representative labels set forth above may be readily accomplished by one of ordinary skill in the art (see Trichothecene Antibody Conjugate, U.S. Pat. No. 4,744,981; Antibody Conjugate, U.S. Pat. No. 5,106,951; Fluorogenic Materials and Labelling Techniques, U.S. Pat. No. 4,018,884; Metal Radionuclide Labelled Proteins for Diagnosis and Therapy, U.S. Pat. No. 4,897,255; and Metal Radionuclide Chelating Compounds for Improved Chelation Kinetics, U.S. Pat. No. 4,988,496; see also Inman, Methods In Enzymology, Vol. 34, Affinity Techniques, Enzyme Purification: Part B, Jakoby and Wichek (eds.), Academic Press, New York, p. 30, 1974; see also Wilchek and Bayer, “The Avidin-Biotin Complex in Bioanalytical Applications,” Anal. Biochem. 171:1-32, 1988).

In some embodiments of the present invention, transferrin, transferrin receptor or antibodies to transferrin receptor are labeled using the techniques generally known in the art and briefly mentioned above.

(b) Experimental Systems

Eukaryotic expression systems can be used for many studies of the p97 gene and protein including to test effectiveness of pharmacological agents, to study the function of the normal complete protein, specific portions of the protein, or of naturally occurring and artificially produced mutant proteins.

Using the techniques known in the art, expression vectors containing the murine p97 cDNA sequence or portions thereof can be introduced into a variety of cells including murine cells and mammalian cells from other species as well as non-mammalian cells. Expression of the murine p97 gene in cell systems may also be used to demonstrate structure-function relationships as well as to provide cell lines for the purposes of drug screening.

The invention also provides methods for examining the function of the murine p97 protein encoded by the nucleic acid molecule of the invention. For example, mp97 may be expressed in non-human transgenic animals such as mice, rats, rabbits, sheep and pigs (see Hammer et al. (Nature 315:680-683, 1985), Palmiter et al. (Science 222:809-814, 1983), Brinster et al. (Proc Natl. Acad. Sci USA 82:44384442, 1985), Palmiter and Brinster (Cell. 41:343-345, 1985) and U.S. Pat. No. 4,736,866). Preferably, the animal is a mouse. The mice used to prepare the transgenic mice can be wild type mice or, alternatively, mice having known phenotypic or genotypic abnormalities. A preferred source is the Alzheimer's Disease model mouse, disclosed elsewhere in this specification. Briefly, an expression unit, including a DNA sequence to be expressed together with appropriately positioned expression control sequences, is introduced into pronuclei of fertilized eggs. Introduction of DNA is commonly done by microinjection. Integration of the injected DNA is detected by blot analysis of DNA from tissue samples, typically samples of tail tissue. It is preferred that the introduced DNA be incorporated into the germ line of the animal so that it is passed on to the animal's progeny. Tissue-specific expression may be achieved through the use of a tissue-specific promoter, or through the use of an inducible promoter, such as the metallothionein gene promoter (Palmiter et al., 1983, ibid), which allows regulated expression of the transgene. Animals which develop tissuespecific expression of mp97 (e.g., in the brain) may be utilized as disease models for Alzheimer's Disease. Alternatively, yeast artificial chromosomes (YACs) may be utilized to introduce DNA into embryo derived stem cells by fusion with yeast spheroblasts carrying the YAC (see Capecchi, Nature 362:255-258, 1993; Jakobovits et al., Nature 362:255-258, 1993). Utilizing such methods, animals may be developed which express mp97 in tissue (e.g. the brain) or at different stages in the development cycle.

Accordingly, the present invention provides a transgenic non-human animal whose germ cells and somatic cells contain a p97 gene introduced into the animal or an ancestor of the animal at an embryonic stage.

The present applicant has previously demonstrated that an elevated level of p97 is diagnostic of Alzheimer's disease (AD). As a result, mice with increased levels of p97 are useful models for studying AD and for testing potential therapies for AD.

Accordingly, the present invention provides a method for screening a therapeutic agent for treating Alzheimer's disease (AD) comprising administering the agent to a mouse having an elevated level of mp97, and measuring the level of mp97 wherein a decrease in levels of mp97 indicates that the agent may be useful in treating Alzheimer's disease. The mouse having increased levels of p97 can be a transgenic mouse as described herein or can be a mouse prone to AD as described by Hsiao et al. (1996). In addition to the transgenic mice, the screening assays can also be performed on transformed cell lines expressing p97.

In addition to animals that express p97, animals which do not produce p97 may be developed in order to study the function of p97. Cells, tissues, and non-human animals lacking in expression or partially lacking in expression of the protein may be developed using recombinant molecules of the invention having specific deletion or insertion mutations in the nucleic acid molecule of the invention. A recombinant molecule may be used to inactivate or alter the endogenous gene by homologous recombination, and thereby create a deficient cell, tissue or animal. Such a mutant cell, tissue or animal may be used to define specific cell populations, developmental patterns and in vivo processes, normally dependent on the protein encoded by the nucleic acid molecule of the invention.

To confirm the role of p97, a p97 knockout mouse can be prepared. By way of example, a targeted recombination strategy may be used to inactivate the endogenous p97 gene. A gene which introduces stop codons in all reading frames and abolishes the biological activity of the protein may be inserted into a genomic copy of the protein. The mutated fragment may be introduced into embryonic stem cells and colonies may be selected for homologous recombination with positive (neomycin)/negative (gancyclovir, thymidine kinase) resistance genes. To establish germ line transmission, two clones carrying the disrupted gene on one allele may be injected into blastocyts of C57/B16 mice and transferred into B6/SJL foster mothers. Chimeras may be mated to C7B1/6 mice and progeny analysed to detect animals homozygous for the mutation (p97−/−).

Accordingly, the present invention provides a transgenic non-human animal having a decreased expression of murine p97. The invention also includes the use of such a transgenic knock-out animal to study p97.

Transgenic p97 mice can be used for a variety of purposes. For example, a p97 knock-out mouse will help identify essential physiological roles for p97 in development and adult functioning of the organism. Once these roles are ascertained, a p97 transgenic mouse can then be used for screening potential therapeutic agents that may act through p97 or p97 related pathways. For example a potential therapeutic agent can be tested in a control mouse and a transgenic p97 mouse to determine if a different response is obtained, thus implicating p97 or the p97 pathway in the activity of the therapeutic agent. Both p97 knock out and p97 knock-in mice are useful in these assessments. Particularly preferred is the use of p97 transgenic Alzheimer's Disease model mice which will reveal if a potential therapeutic agent that is useful in treating the Alzheimer's Disease phenotype is in any way enhanced or diminished in the p97 transgenic variant. This in turn can lead to novel and improved therapeutic agents.

An alternate use for a p97 transgenic mouse is for testing of potential therapeutic agents and diagnostic agents that are conjugated to p97 protein as disclosed in section (a) herein. Compositions such as p97-adriamycin or p97-taxol conjugates may have different effects depending on whether the host mouse produces no endogenous p97 (p97 knock-out) or whether it has p97 overexpression (p97 knock-in). In this regard, use of mouse p97-conjugates may be preferred for testing in transgenic p97 mice, as compared to the human p97-conjugates.

(c) Antisense Oligonucleotides

Isolation of a nucleic acid molecule encoding the murine p97 enables the production of antisense oligonucleotides that can modulate the expression and/or activity of p97.

Accordingly, the present invention provides an antisense oligonucleotide that is complimentary to a nucleic acid sequence encoding p97.

The term “antisense oligonucleotide” as used herein means a nucleotide sequence that is complimentary to its target.

The term “oligonucleotide” refers to an oligomer or polymer of nucleotide or nucleoside monomers consisting of naturally occurring bases, sugars, and intersugar (backbone) linkages. The term also includes modified or substituted oligomers comprising non-naturally occurring monomers or portions thereof, which function similarly. Such modified or substituted oligonucleotides may be preferred over naturally occurring forms because of properties such as enhanced cellular uptake, or increased stability in the presence of nucleases. The term also includes chimeric oligonucleotides which contain two or more chemically distinct regions. For example, chimeric oligonucleotides may contain at least one region of modified nucleotides that confer beneficial properties (e.g. increased nuclease resistance, increased uptake into cells), or two or more oligonucleotides of the invention may be joined to form a chimeric oligonucleotide.

The antisense oligonucleotides of the present invention may be ribonucleic or deoxyribonucleic acids and may contain naturally occurring bases including adenine, guanine, cytosine, thymidine and uracil. The oligonucleotides may also contain modified bases such as xanthine, hypoxanthine, 2-aminoadenine, 6-methyl, 2-propyl and other alkyl adenines, 5-halo uracil, 5-halo cytosine, 6-aza uracil, 6-aza cytosine and 6-aza thymine, pseudo uracil, 4-thiouracil, 8-halo adenine, 8-aminoadenine, 8-thiol adenine, 8-thiolalkyl adenines, 8-hydroxyl adenine and other 8-substituted adenines, 8-halo guanines, 8-amino guanine, 8-thiol guanine, 8-thiolalkyl guanines, 8-hydroxyl guanine and other 8-substituted guanines, other aza and deaza uracils, thymidines, cytosines, adenines, or guanines, 5-trifluoromethyl uracil and 5-trifluoro cytosine.

Other antisense oligonucleotides of the invention may contain modified phosphorous, oxygen heteroatoms in the phosphate backbone, short chain alkyl or cydoalkyl intersugar linkages or short chain heteroatomic or heterocyclic intersugar linkages. For example, the antisense oligonucleotides may contain phosphorothioates, phosphotriesters, methyl phosphonates, and phosphorodithioates. In an embodiment of the invention there are phosphorothioate bonds links between the four to six 3′-terminus bases. In another embodiment phosphorothioate bonds link all the nucleotides.

The antisense oligonucleotides of the invention may also comprise nucleotide analogs that may be better suited as therapeutic or experimental reagents. An example of an oligonucleotide analogue is a peptide nucleic acid (PNA) wherein the deoxyribose (or ribose) phosphate backbone in the DNA (or RNA), is replaced with a polyamide backbone which is similar to that found in peptides (P. E. Nielsen, et al Science 1991, 254, 1497). PNA analogues have been shown to be resistant to degradation by enzymes and to have extended lives in vivo and in vitro. PNAs also bind stronger to a complimentary DNA sequence due to the lack of charge repulsion between the PNA strand and the DNA strand. Other oligonucleotides may contain nucleotides containing polymer backbones, cyclic backbones, or acyclic backbones. For example, the nucleotides may have morpholino backbone structures (U.S. Pat. No. 5,034,506). Oligonucleotides may also contain groups such as reporter groups, a group for improving the pharmacokinetic properties of an oligonucleotide, or a group for improving the pharmacodynamic properties of an antisense oligonucleotide. Antisense oligonucleotides may also have sugar mimetics.

The antisense nucleic acid molecules may be constructed using chemical synthesis and enzymatic ligation reactions using procedures known in the art. The antisense nucleic acid molecules of the invention or a fragment thereof, may be chemically synthesized using naturally occurring nucleotides or variously modified nucleotides designed to increase the biological stability of the molecules or to increase the physical stability of the duplex formed with mRNA or the native gene e.g. phosphorothioate derivatives and acridine substituted nucleotides. The antisense sequences may be produced biologically using an expression vector introduced into cells in the form of a recombinant plasmid, phagemid or attenuated virus in which antisense sequences are produced under the control of a high efficiency regulatory region, the activity of which may be determined by the cell type into which the vector is introduced.

The antisense oligonucleotides may be introduced into tissues or cells using techniques in the art including vectors (retroviral vectors, adenoviral vectors and DNA virus vectors) or physical techniques such as microinjection. The antisense oligonucleotides may be directly administered in vivo or may be used to transfect cells in vitro which are then administered in vivo. In one embodiment, the antisense oligonucleotide may be delivered to macrophages and/or endothelial cells in a liposome formulation.

(d) Murine p97 Modulators

In addition to antibodies and antisense oligonucleotides described above, other substances that modulate p97 expression or activity may also be identified. Accordingly, the present invention includes the use of the nucleic acids encoding murine p97 and the p97 protein to develop or identify substances that modulate murine p97 expression or activity.

(i) Substances that Bind Murine p97

Substances that affect murine p97 activity can be identified based on their ability to bind to murine p97.

Substances which can bind with the murine p97 of the invention may be identified by reacting the murine p97 with a substance which potentially binds to murine p97, and assaying for complexes, for free substance, or for non-complexed murine p97, or for activation of murine p97. In particular, a yeast two hybrid assay system may be used to identify proteins which interact with murine p97 (Fields, S. and Song, O., 1989, Nature, 340:245-247). Systems of analysis which also may be used include ELISA.

Accordingly, the invention provides a method of identifying substances which can bind with murine p97, comprising the steps of:

(a) reacting murine p97 and a test substance, under conditions which allow for formation of a complex between the murine p97 and the test substance, and

(b) assaying for complexes of murine p97 and the test substance, for free substance or for non complexed murine p97, wherein the presence of complexes indicates that the test substance is capable of binding murine p97.

The murine p97 protein used in the assay may have the amino acid sequence shown in SEQ.ID.NO.: 2 or may be a fragment, analog, derivative, homolog or mimetic thereof as described herein.

Conditions which permit the formation of substance and murine p97 complexes may be selected having regard to factors such as the nature and amounts of the substance and the protein.

The substance-protein complex, free substance or non-complexed proteins may be isolated by conventional isolation techniques, for example, salting out, chromatography, electrophoresis, gel filtration, fractionation, absorption, polyacrylamide gel electrophoresis, agglutination, or combinations thereof. To facilitate the assay of the components, antibody against murine p97 or the substance, or labelled murine p97, or a labelled substance may be utilized. The antibodies, proteins, or substances may be labelled with a detectable substance as described above.

Murine p97, or the substance used in the method of the invention may be insolubilized. For example, murine p97 or substance may be bound to a suitable carrier. Examples of suitable carriers are agarose, cellulose, dextran, Sephadex, Sepharose, carboxymethyl cellulose polystyrene, filter paper, ion-exchange resin, plastic film, plastic tube, glass beads, polyamine-methyl vinyl-ether-maleic acid copolymer, amino acid copolymer, ethylene-maleic acid copolymer, nylon, silk, etc. The carrier may be in the shape of, for example, a tube, test plate, beads; disc, sphere etc.

The insolubilized protein or substance may be prepared by reacting the material with a suitable insoluble carrier using known chemical or physical methods, for example, cyanogen bromide coupling.

The p97 proteins or substance may also be expressed on the surface of a cell using the methods described herein.

The invention also contemplates assaying for an antagonist or agonist of the action of murine p97.

It will be understood that the agonists and antagonists that can be assayed using the methods of the invention may act on one or more of the binding sites on the protein or substance including agonist binding sites, competitive antagonist binding sites, non-competitive antagonist binding sites or allosteric sites.

The invention also makes it possible to screen for antagonists that inhibit the effects of an agonist of murine p97. Thus, the invention may be used to assay for a substance that competes for the same binding site of murine p97.

(ii) Peptide Mimetics

The present invention also includes peptide mimetics of the murine p97 of the invention. For example, a peptide derived from a binding domain of murine p97 will interact directly or indirectly with an associated molecule in such a way as to mimic the native binding domain. Such peptides may include competitive inhibitors, enhancers, peptide mimetics, and the like. All of these peptides as well as molecules substantially homologous, complementary or otherwise functionally or structurally equivalent to these peptides may be used for purposes of the present invention.

“Peptide mimetics” are structures which serve as substitutes for peptides in interactions between molecules (See Morgan et al (1989), Ann. Reports Med. Chem. 24:243-252 for a review). Peptide mimetics include synthetic structures which may or may not contain amino acids and/or peptide bonds but retain the structural and functional features of a peptide, or enhancer or inhibitor of the invention. Peptide mimetics also include peptoids, oligopeptoids (Simon et al (1972) Proc. Natl. Acad, Sci USA 89:9367); and peptide libraries containing peptides of a designed length representing all possible sequences of amino acids corresponding to a peptide of the invention.

Peptide mimetics may be designed based on information obtained by systematic replacement of L-amino acids by D-amino acids, replacement of side chains with groups having different electronic properties, and by systematic replacement of peptide bonds with amide bond replacements. Local conformational constraints can also be introduced to determine conformational requirements for activity of a candidate peptide mimetic. The mimetics may include isosteric amide bonds, or D-amino acids to stabilize or promote reverse turn conformations and to help stabilize the molecule. Cyclic amino acid analogues may be used to constrain amino acid residues to particular conformational states. The mimetics can also include mimics of inhibitor peptide secondary structures. These structures can model the 3-dimensional orientation of amino acid residues into the known secondary conformations of proteins. Peptoids may also be used which are oligomers of N-substituted amino acids and can be used as motifs for the generation of chemically diverse libraries of novel molecules.

Peptides of the invention may also be used to identify lead compounds for drug development. The structure of the peptides described herein can be readily determined by a number of methods such as NMR and X-ray crystallography. A comparison of the structures of peptides similar in sequence, but differing in the biological activities they elicit in target molecules can provide information about the structure-activity relationship of the target. Information obtained from the examination of structure-activity relationships can be used to design either modified peptides, or other small molecules or lead compounds that can be tested for predicted properties as related to the target molecule. The activity of the lead compounds can be evaluated using assays similar to those described herein.

Information about structure-activity relationships may also be obtained from co-crystallization studies. In these studies, a peptide with a desired activity is crystallized in association with a target molecule, and the X-ray structure of the complex is determined. The structure can then be compared to the structure of the target molecule in its native state, and information from such a comparison may be used to design compounds expected to possess.

(e) Drug Screening Methods

In accordance with one embodiment, the invention enables a method for screening candidate compounds for their ability to increase or decrease the activity of a murine p97 protein. Such compounds may have therapeutic utility for example in treating Alzheimer's disease. The method comprises providing an assay system for assaying p97 activity, assaying the activity in the presence or absence of the candidate or test compound and determining whether the compound has increased or decreased p97 activity.

Accordingly, the present invention provides a method for identifying a compound that affects murine p97 protein activity or expression comprising:

(a) incubating a test compound with a murine p97 protein or a nucleic acid encoding a murine p97 protein; and

(b) determining an amount of murine p97 protein activity or expression and comparing with a control (i.e. in the absence of the test substance), wherein a change in the murine p97 protein activity or expression as compared to the control indicates that the test compound has an effect on murine p97 protein activity or expression.

In accordance with a further embodiment, the invention enables a method for screening candidate compounds for their ability to increase or decrease expression of a p97 protein. The method comprises putting a cell with a candidate compound, wherein the cell includes a p97 gene or portion thereof operably joined to a reporter gene coding region, and detecting a change in expression of the reporter gene.

In one embodiment, the present invention enables culture systems in which cell lines which express the p97 gene, and thus p97 protein products, are incubated with candidate compounds to test their effects on p97 expression. Such culture systems can be used to identify compounds which upregulate or downregulate murine p97 expression or its function, through the interaction with other proteins.

Such compounds can be selected from protein compounds, chemicals and various drugs that are added to the culture medium. After a period of incubation in the presence of a selected test compound(s), the expression of p97 can be examined by quantifying the levels of p97 mRNA using standard Northern blotting procedure, to determine any changes in expression as a result of the test compound. Cell lines transfected with constructs expressing p97 can also be used to test the function of compounds developed to modify the protein expression. In addition, transformed cell lines expressing a normal p97 protein could be mutagenized by the use of mutagenizing agents to produce an altered phenotype in which the role of mutated p97 can be studied in order to study structure/function relationships of the protein products and their physiological effects.

Animal models are also important for testing novel drugs and thus may also be used to identify any potentially useful compound affecting p97 expression and activity and thus physiological function. Animal models containing increased or decreased expression of p97 have been previously described herein.

These and other aspects of the present invention will become evident upon reference to the following detailed examples, which are intended to illustrate, but not limit, the scope of the invention. In addition, reference is made herein to various patents and publications, which are hereby incorporated by reference in their entirety.

The following non-limiting examples are illustrative of the present invention:

### EXAMPLES

### Example 1

Cloning of mp97

Identification of Polynucleotide Fragments in Mouse EST Database

The human p97 sequence was used to search the mouse EST databaseS for any existing clones which had significant homology with human p97. At the time the homology search was first performed, the longest mp97 EST available was the IMAGE clone mf07c08.r1. The clone was ordered from ATCC (American Type Cell Cultures) and its entire sequence was determined. The cDNA is about 2.4 kb in size and corresponds to the C-terminal half of the mp97 lacking about half of the coding region and the 5′ untranslated leader sequence. The known p97 sequences were used for making primers to clone the missing 5′ portion of the cDNA by RT-PCR methods described below. The primers used are given in Table 1.

TABLE 1
Oligonucleotides Used in the Cloning of Mp97

| | |
|---|---|
| mMTf + 1 | GAC TCA AGC TTG CCA GCT GCG TGC CTG TC |
| mMTf + 2 | GTG GTG GCT GTG GCT AGA A |
| mMTf + 3 | TTC CCA ACA TCA CCA ACG C |
| mMTf + 4 | CTG GAC AAG GCC CAG GAC CTG |
| mMTf + 5 | TGA GGG AGA GGC AAG GTG |
| mMTf + 6 | GCC AGA GCT GTA CTG TGG |
| mMTf + 7 | CTT ATC CGT GTG AAC ATA TCT G |
| mMTf + 8 | TGG AGA CGT TGC GAG CTG |
| mMTf + 9 | TCT GTC GCC TCT GCC GTG |
| mMTf − 1 | GTC AAG GAT CCG AAG GCC ACA GCC ATA TCT C |
| mMTf − 2 | GCG TTG GTG ATG TTG GGA A |
| mMTf − 3 | TTC TAG CCA CAG CCA CCA C |
| mMTf − 4 | GCT CCT ACT TCT TCA GAC AAG CAG |
| mMTf − 5 | TGC ATG CTC CAC AAG GCA CCT GAA GG |
| mMTf − 6 | CAG GTC CTG GGC CTT GTC CAG |
| mMTf − 7 | CCA CAG TAC AGC TCT GGC |
| mMTf − 8 | CAC CTT GCC TCT CCC TCA |
| mMTf − 9 | AGG CAC AGG TTC GCT GCT G |
| mMTf − 10 | AGC AGC GGT CTT CAG AGA |
| mMTf − 11 | GCT GGA AGT CCT CTG ACA |
| mMTf − 12 | GTG CTA GCT AGC GCT CTG CGT CTG AGA TGG |
| pME18S 5′ | CTT CTG CTC TAA AAG CTG CG |
| pME18S 3′ | CGA CCT GCA GCT CGA GCA CA |

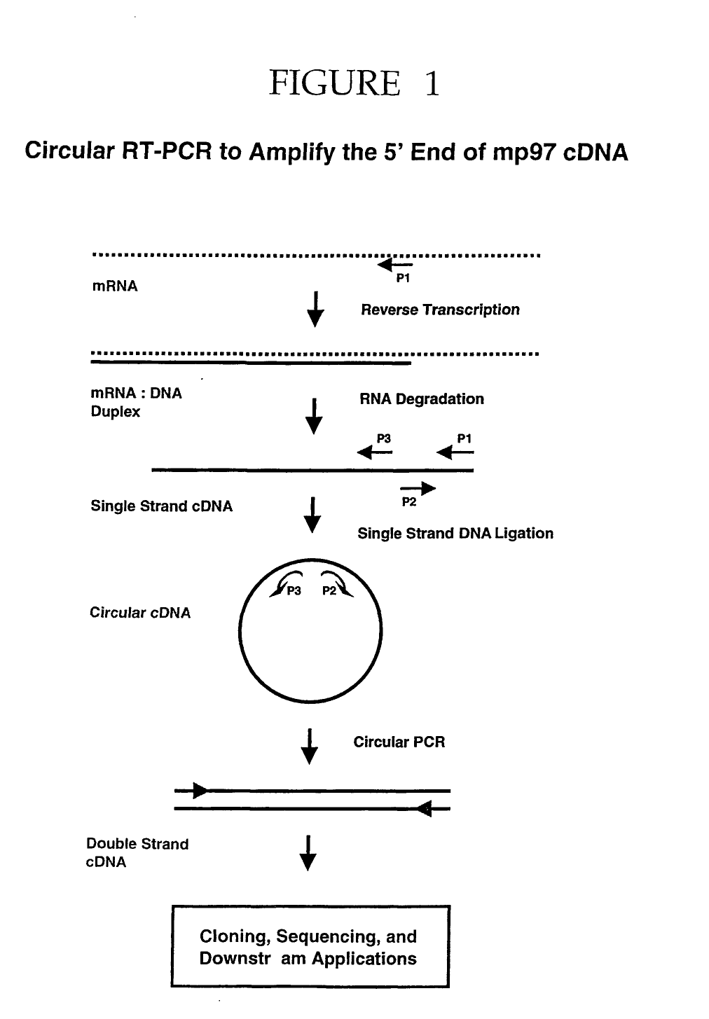

Extending the mf07c08.r1 EST Clone by Circular RT-PCR (FIG. 1)

1. JB/MS Mouse Melanoma Cell Line

The mouse melanoma cell line JB/MS was chosen for RNA isolation because it is known to express the mp97 protein. The cells were cultured in 98 mm tissue culture petri dishes according to standard cell culture procedures. The medium used was DMEM supplemented with L-glutamine, Hepes and non-essential amino acids.

2. Total RNA Isolation

The JB/MS cells were harvested from the tissue culture dishes by treating with 0.25% trypsin and monitoring cell detachment under a microscope. The cells were pelleted by spinning at 1,100 rpm for 5 minutes and lysed by adding and mixing with 6 ml of GITC lysis buffer. A CsCl cushion was prepared by adding 4 ml of CsCl to a 12 ml ultracentrifuge tube. The cell lysate was layered over the CsCl cushion. The tubes were centrifuged at 32,000 rpm for 16 hours at room temperature, the supernatants removed, and the pellets air dried in the tubes. The RNA-containing pellet was dissolved in 200 ul of distilled water followed by ethanol precipitation. The final RNA pellet was dissolved in 200 ul of distilled and deionized water.

3. Poly A+ mRNA Isolation

The poly A+ mRNA was isolated using the Promega PolyATtract mRNA Isolation System III following the manufacturer's instruction.

4. mp97 Specific Reverse Transcription

The primer used for reverse transcription was mMTf-5: TGCATGCTCCACAAGGCACCTGAAGG. Two hundred ng of mMTf-5 was mixed with 80 ng of the JB/MS poly A+ mRNA, and dH2O in final volume of 12 ul in microfuge tubes. The mixture was heated at 70° C. for 10 minutes to denature the RNA, followed by quick chill on ice. Four ul of 5× First Strand Buffer were added to 2 ul of 0.1M DTT, and 1 ul of 10 mM dNTP mix. The contents of the tubes was were prewarmed at 42° C. for 2 min, followed by the addition of 200 units of SuperScript II (GIBCO BRL). The tubes were incubateincubated at 42° C. for one hour and the reaction was stopped by heating at 75° C. for 15 min. The RNA was removed by incubating with 2 units of RNaseH at 37° C. for 20 min.

5. Single Strand cDNA Ligation

A 50 ul ligation reaction was set up as follows: 50 mM of Hepes pH 7.4, 10 mM of MgCl2, 5 mM of DTT, 5 ul of the first strand cDNA, 2 mM of ATP, 26 units of T4 RNA ligase. The ligation reaction was incubated at 17° C. overnight.

6. Circular PCR

The strategy used is shown schematically in FIG. 1. The primer pair used was:

mMTf+1 GACTCAAGCTTGCCAGCTGCGTGCCTGTC, corresponding to the mp97 coding strand with a HindIII adaptor., Tm=64° C., and mMTf−1 GTCAAGGATCCGAAGGCCACAGCCATATCTC, corresponding to the non-coding strand with a Bami adapter., Tm=62° C.

PCR reaction: The reaction was set up in 100 ul volume that contained sterile distilled water, 1×Pfu buffer, 0.2 mM of dNTP's, 1 ul of the ligated single strand cDNA, 0.26 pmole of both primers, and 5 units of cloned Pfu DNA polymerase (Stratagene). The amplification was carried out for 30 cycles at: 94° C. for 30 seconds, 57° C. for 30 seconds, and 72° C. for 4 min.

7. Cloning the PCR product

Gel purification of the PCR product: A portion of the PCR reaction was loaded on a 0.7% agarose gel. A 0.8 kb fragment was amplified without any significant nonspecific product. The fragment was purified using the QIAEX II gel extraction kit (QIAGEN) following the supplier's instructions.

Restriction digestion: 23 ng of the purified PCR product and 1 ug of pBluescript (KS+) phagemid DNA were digested with both BamHI and HindIII restriction enzymes at 37 C. for 1 hour.

DNA purification: The digested PCR product was extracted twice with chloroform followed by ethanol precipitation. The cut phagemid DNA was gel purified as above using the QIAEX II gel extraction kit.

Ligation reaction was set up in a final volume of 12 ul by mixing together sterile distilled water, the purified 0.8 kb fragment and pBluescript II, 10× ligation buffer and 1.5 units of T4 DNA ligase. The reaction was incubated at 16° C. overnight.

Transformation: 1 ul of the ligation mix was used for electroporation to transform the host bacterium DH10B. The transformed bacteria were grown in LB medium at 37° C. for 1 hour and then spread on LB+Ampicillin+IPTG+X-Gal plates and incubated at 37° C. overnight. Two white colonies were selected for plasmid DNA preparation.

Plasmid DNA isolation was carried out using the Wizard DNA Isolation kit (Promega) following the Manufacturer's instruction. Both clones had an insert with the expected size of 0.8 kb when digested with BamHI and HindIII.

Determination of the Mouse p97Mp97 cDNA Sequence

All DNA sequencing was performed at the Nuicleic Acid/Protein Services (NAPS) Unit of the Biotechnology Lab in the University of British Columbia. The DNA templates used were the 0.8 kb circular RT-PCR product or cloned plasmid, and the two EST clones. The cycle sequencing reactions were carried out with PE Applied Biosystems BigDye terminator premix using a Perkin-Elmer thermal cycler as follows: 4 ul of premix, 500 ng of plasmid DNA or 90 ng of PCR product, 3.2 pmole of the primer in 20 ul volume. The temperature cycling consisted of the following:

Rapid thermal ramp to 96° C.

96° C. for 30 seconds

Rapid thermal ramp to 50° C.

50° C. for 15 seconds

Rapid thermal ramp to 60° C.

60° C. for 4 minutes

25 cycles total

Rapid thermal ramp to 4° C. (soak file) and hold.

The reactions were purified by ethanol precipitation and sequenced on a Perkin-Elmer Model 480 machine. The raw sequence data were edited and compiled using the DNASIS sequence analysis software (Hitachi Software) and/or other on line DNA sequence analysis programs. To date, a composite of 3,937 bp mp97 cDNA sequence have been determined using the three mp97 cDNA clones. The cDNA sequence is presented in SEQ.ID.NO.: 1 and the predicted protein sequence is presented in SEQ.ID.NO.: 2.

### Example 2

Expression of a Truncated mp97 Protein

The Bacterial Expression System pGEX and Generation of a mp97 Protein Expression Construct

The pGEX system was chosen due to its inducible high level expression and the ease of affinity purification of the expressed product. The cloned cDNA produces a fusion protein with the glutathione S-transferase (GST) at the N-terminal. GST has a high affinity to the tripeptide glutathione (gamma-Glu-Cys-Gly), which is used for affinity purification of the fusion protein. There is an engineered thrombin cleavage site at the fusion junction that can be used to cleave the protein of interest off from GST.

The longest mp97 EST at the time, mf07c08.r1, was used for expression in the pGEX system (Pharmacia). The DNA was cloned in the vector pT7T3D (Pharmacia) at EcoRI and NotI site. The 5′ EcoRI site of the EST happens to be in frame with the GST coding region of pGEX-4T-1 when fused with its EcoRI site. Thus pGEX4T-1 was chosen for expression. EcoRI and NotI double digestion: about 4 ug each of mf07c08.r1 and pGEX-4T-1 DNA were digested with both EcoRI and NotI enzymes for 1.5 hr. at 37° C. The digested DNA were extracted once with phenol/chloroform and once with chloroform only, followed by ethanol precipitation. The extracted DNA were loaded on a 0.7% agarose gel and the 2.4 kb EST and the linearized pGEX excised for purification. The gel purification of the DNA were carried out with the QIAEX II gel extraction kit (QIAGEN) following the supplier's instruction. The DNA were eluted in a final volume of 25 ul.

The ligation reaction was set up as follows: 6 ul of EST+6 ul of pGEX+1.5 ul of 10× buffer+1.5 ul of T4 DNA ligase. The reaction was incubated at 16° C. overnight. 1 ul of the ligation reaction was used to transform the electrocompetent bacterium DH10B. Electroporation was performed using a EC100 electroporator of E-C Apparatus Corporation following the manufacturer's instruction. The transformed cell were plated on LB+ampicillin plates. 10 individual colonies were selected for plasmid DNA preparation to screen for positive clones. All 10 plasmid DNA, when digested with both EcoRI and NotI, released an identical insert of about 2.4 kb.

Expression and Purification of the GST-p97mp97 Fusion Protein

Bacterial culture: 100 ml of LB+ampicillin was innoculated with an individual pGEX-mp97 colony and incubated at 37° C. overnight. On the next day, 1 liter of LB+ampicillin was innoculated with 50 ml of the overnight culture (1:20 dilution) and continued to grow at 37° C. for 100 min. To induce expression, 0.5 M IPTG was added to a final concentration of 0.15 mM, and the cultures were incubated at 37° C. for a further 3 hr.

Affinity purification: The bacteria were pelleted at 5,000 g for 10 min, the supernatant removed and the pellets were frozen at −80° C. overnight. The pellets were resuspended in a total of 20 ml of ice cold PBS and sonicated to lyse the bacteria using a Branson Sonifier 450 and a 5 mm probe, at setting 3 for 3×15 seconds. Triton X-100 was added to the lysate to a final concentration of 1% to lyse the cells. Tubes containing the lysate were centrifuged at 10,000 g for 5 min. to remove cell debris, and the supernatant transferred to a clean 50 ml tube. One ml of glutathione-cross linked beaded agarose (Sigma, rehydrated and washed following the supplier's instruction) were added. The tubes were gently rocked at room temperature to mix the contents. The agarose beads were spun down at 1,000 g for 30 seconds, and washed 3 times with 50 ml of ice cold PBS by resuspending and spinning at 1,000 g. The GST-mp97 fusion protein was eluted by mixing with 1 ml of 50 mM Tris.HCl (pH8.0)+5 mM reduced glutathione, rocking at room temperature for 5 min. and spinning at 1,000 g for 30 seconds. The elution step was repeated four more times. The eluted fusion protein was characterized by SDS-PAGE and had an apparent molecular weight of about 63 kDa (including about 27 kDa of GST).

Cleavage and purification of the mp97 protein from GST: The fusion protein was cleaved with 0.5 unit/10 ul of thrombin by incubating at 30° C. for 2 hr. The cleaved GST was depleted from the mixture with glutathione conjugated agarose beads following procedures described above.

### Example 3

Production of Polyclonal Antibodies Against mp97 Protein

Two New Zealand White rabbits were immunized with 100 ug of purified mp97 protein and subsequently boosted every month for three more times months. The immunizations were done with injections at multiple locations as follows: first immunization, lymph node and intravenous injections; second immunization, sub-scap and intramuscular injections; third immunization, lymph node and intravenous injections; final bleed. Ten days after each injection, 30-35 ml of blood were taken to test the antibody titer. The blood was first heated at 37° C. for one hr with occasional stirring to detach it from the tube and then left at 4° C. overnight before the serum was collected. The serum was then filtered through a 0.2 uM sterile filtration disc and stored at −80° C. The serum was tested for antibody titer against the expressed mp97 fragment cleaved from the GST fusion described above.

While the present invention has been described with reference to what are presently considered to be preferred examples, it is to be understood that the invention is not limited to the disclosed examples. To the contrary, the invention is intended to cover various modifications and equivalents included within the spirit and scope of the appended claims.

All publications, patents and patent applications are herein incorporated by reference in their entirety to the same extent as if each individual publication, patent or patent application was specifically and individually indicated to be incorporated by reference in its entirety.

| | |
|---|---|
| DNASIS | |
| SEQ | |
| TCGCCTACTG GGAAGAGGAA GCCAGGACAG ACCCGCCAGC ACCCCAGCCA ACCCAACGTT | SEQ.ID.NO.:1 |
| GCCATGAGGC TCCTGAGCGT GACTTTTTGG CTACTCCTGT CCCTGCGCAC TGTCGTCTGT | |
| GTGATGGAGG TGCAGTGGTG TACCATCTCA GACGCAGAGC AGCAGAAGTG CAAAGACATG | |
| AGCGAGGCCT TCCAGGGAGC TGGCATTCGT CCTTCCCTTC TCTGCGTCCA GGGCAACTCC | |
| GCTGACCACT GTGTCCAGCT CATCAAGGAA CAAAAAGCAG ATGCCATCAC CCTGGATGGA | |
| GGGGCCATCT ATGAGGCAGG GAAGGAGCAC GGCCTGAAGC CAGTGGTGGG GGAAGTCTAT | |
| GACCAAGACA TTGGGACTTC CTATTATGCC GTGGCTGTGG TCAGGAGGAA TTCCAATGTT | |
| ACCATCAACA CCCTGAAGGG CGTCAAGTCC TGCCACACAG GCATTAACCG GACTGTGGGC | |
| TGGAACGTGC CTGTCGGTTA CCTCGTAGAG AGCGGCCATC TGTCAGTGAT GGGCTGTGAT | |
| GTGCTCAAAG CCGTTGGTGA TTATTTTGGA GGCAGCTGTG TCCCTGGAAC AGGAGAAACC | |
| AGCCATTCCG AGTCCCTCTG TCGCCTCTGC CGTGGCGACT CTTCTGGGCA CAATGTGTGT | |
| GACAAGAGTC CCCTAGAGAG ATACTACGAC TACAGTGGAG CCTTCCGGTG CCTGGCGGAA | |
| GGAGCCGGTG ACGTGGCCTT CGTGAAGCAC AGCACAGTGC TGGAAAATAC TGATGGAAAC | |
| ACCCTGCCTT CCTGGGGCAA GTCCCTGATG TCAGAGGACT TCCAGCTACT ATGCAGGGAT | |
| GGCAGCCGAG CCGACATCAC TGAGTGGAGA CGTTGCCACC TGGCCAAGGT GCCTGCTCAT | |
| GCTGTGGTGG TCAGGGGTGA CATGGATGGC GGTCTCATAT TCCAACTGCT CAACGAAGGC | |
| CAGCTTCTGT TCAGCCAYGA AGACAGCAGC TTCCAGATGT TCAGCTCCAA AGCCTACAGC | |
| CAGAAGAACT TGCTGTTCAA AGACTCCACC TTGGAGCTTG TGCCCATTGC CACACAGAAC | |
| TATGAGGCCT GGCTGGGCCA GGAATACCTG CAGGCCATGA AGGGGCTCCT CTGTGATCCC | |
| AACCGGCTGC CCCACTACCT GCGCTGGTGT GTGCTGTCAG CGCCCGAGAT CCAGAAGTGT | |
| GGAGATATGG CTGTGGCCTT CAGCCGCCAG AATCTCAAGC CGGAAATTCA GTGTGTGTCG | |
| GCCGAGTCCC CTGAGCACTG CATGGAGCAG ATCCAGGCTG GGCACACTGA CGCTGTGACT | |
| CTGAGGGGCG AGGACATTTA CAGGGCAGGA AAGGTGTACG GCCTGGTTCC GGCGGCCGGG | |
| GAGCTGTATG CTGAGGAGGA CAGGAGCAAT TCCTACTTTG TGGTGGCTGT GGCAAGAAGG | |
| GACAGCTCCT ACTCCTTCAC CCTGGACGAG CTTCGCGGCA AGCGTTCCTG CCACCCCTAC | |
| TTGGGCAGCC CAGCGGGCTG GGAGGTGCCC ATCGGCTCCC TCATCCAGCG GGGCTTCATC | |
| CGGCCCAAGG ACTGTGATGT CCTCACAGCG GTGAGCCAGT TCTTCAATGC CAGCTGCGTG | |
| CCTGTCAACA ACCCTAAGAA CTACCCTTCC GCACTATGTG CGCTCTGCGT GGGAGACGAG | |
| AAGGGCCGCA ACAAATGTGT GGGGAGCAGC CAGGAGAGAT ACTACGGCTA CAGCGGGGCC | |
| TTCAGGTGCC TTGTGGAGCA TGCAGGGGAC GTGGCTTTCG TCAAGCACAC GACTGTCTTT | |
| GAGAACACAA ATGGTCACAA TCCTGAGCCT TGGGCTTCTC ACCTCAGGTG GCAAGACTAT | |
| GAACTACTGT GCCCCAATGG GGCACGGGCT GAGGTAGACC AGTTCCAAGC TTGCAACCTG | |
| GCACAAATGC CATCCCACGC TGTCATGGTC CGTCCAGACA CCAACATCTT CACTGTGTAT | |
| GGACTTCTGG ACAAGGCCCA GGACCTGTTT GGAGACGACC ATAACAAGAA CGGTTTCCAA | |
| ATGTTTGACT CCTCCAAATA TCACAGCCAA GACCTGCTTT TCAAAGATGC TACAGTCCGA | |
| GCGGTGCCAG TCCGGGAGAA AACCACATAC CTGGACTGGC TGGGTCCTGA CTATGTGGTT | |
| GCGCTGGAGG GGATGTTGTC TCAGCAGTGC TCCGGTGCAG GGGCCGCGGT CGAGCGAGTC | |
| CCCCTGCTGG CCCTGCTCCT GCTGACCCTG GCTGCAGGCC TCCTTCCTCG CGTTCTCTGA | |
| AGACCGCTGC TTCAGGCCAC GCCCAGAGCA GGGAAAGCTA CACAGCTCAA CCGGAAGAAA | |
| CCAGGACATC AGCTAACCCT GCAGGAGAGC GCGGGGCGGG ATGAGGAGAG GCAAGGTGAG | |
| AACTCACACA CACACACAAG CCTCCGAGGT GCGATTCTAA CCCAAAGAGA AATTTCTAGA | |
| ATCAGGATGA TTGTTAAGGC CAAGTCTTCC CACTTGCTGG AGCCCTCAAT ACCTGAGGCG | |
| ACTGGCGAGT ACGCCAGTCA CTCCTCCCAC ACCGGTGGCG CCAGCAGCGA ACCTGTGCCT | |
| CCCACCTGGA GCCTCCTGGC TGGCTGGGGT GGTTAAGGGG GGGGGGGGGA GAGTGAAGAT | |
| GCTGGTTGCC ATGGCAACCG TGGAGCAGCT TCCAGCCTCT GTACCGGCCA CCTGGTGAGA | |
| TGCCAAGGAA GGAGCACACC ACCAACCTAG GGAACCTGTG CGACACACTA CCACCCAGCA | |
| GCCCCTGCTT TCGCTGCCCC ACCGCTCTTT CCTATGGGCA CTTGTCCACC AAGGCCACAC | |
| CGTCGGAGGG GCAAGGCTGC TGAGCACATC AGCCTTCTGA TGTGACACCA ACCAAGGAGC | |
| CCAGCCCTCT GGACAGCAAG TTTTGCTAGA CTGGGATGGG AGGAAGGCCA GAGCTGTACT | |
| GTGGGGATGA AGTCCTCCAA AACCTCAGAG GAAGGAAGTG CCCCCACCTT CCCATTAAGA | |
| ATGTTAGTGT GTGAGAAACT TGATGCAGGG TGGAAACTAT CCTGTTTAAC GGCTCCCGTG | |
| GCAAGCAGGA CTTGCGCTGT CTGCGCTGCC TGGACCTCAC TGCACAATGA AACTGTTGCC | |
| GAGATTCTAT TGTTTGCTCT CCTGGTCTCA GTCTCAACAT TAGTTTTCTC CCTGCCTTCA | |
| TATACCCCTT CCCACATCAC CACGCAAGCA CGCACGCGCA CACGCACACG CACACACCTT | |
| ATCCGTGTGA ACATATCTGA ACATATCTGC TTGTCTGAAG AAGTAGGAGC TAACCCAAAA | |
| TAACTTCCTG TCATGAGCTG GGCCTTGGGA TATACCACGA GCCAGGGGAT TGGGGAGAGC | |
| CCTGTCTTCC CTTCACCCTG CACCTGTTGG GCAGTTGCAT CTTTCGAGAG GATCCCTGGT | |
| TCTCTCGAAC TGTGAGAGCC AAGGCCTAGG CTGCCATTTT GCCATTGTTC TCTCGAGAAC | |
| CAGAAAAAGT TTTCCAAAGC TACCAGCTCT TACCCCAGAT CTTGTTCCCT TAAAAAAAAG | |
| TAATAAATAA AAAGGAGAAG AAACAGGAGC AAACAGCCAT CGTCAGCACA CTGGAAGCAG | |
| CGTGGGCCGG GAGCTATTTG TGTCTTGGTC TGTGTGGGGG GCCTCAGATC CCAATGACAG | |
| GCCAGGTTCC CAGTGGCTCG CCCCCACCTG TGGGCGACGA CGGGACAGAT CCTTTCCATG | |
| GCTCACCAGT AGAGAAGGTC CTGGCAGTGT CCCAGCCAGA GTCACACAAT CCTGAGGAAA | |
| ATCGGTCACC ATGGTGCTTG GGAGAGCAAG CCCCTCCTCC TCCCAGTACA CAGCCATCCA | |
| TTCTTCTCTG AGCTGGGGAC TTCACAGTGA GAAGTGTACT CTGTGTGGGC GACTGTGCTG | |
| CCCAAAGTGT GATGTCTGTG CCGTGTGCCT TTCAGGTGTG ACTTTGAAGA GCGTTGTGTA | |
| AATGACGTCT GATTGCCATG GGCCACTGCT GTGTTTGTGC TAAAGAAAGA CATTGGTTTC | |
| TTTTTAAAAT AAAGCCATAT ATCCCTGCAA AAAAAAAAAA AAAAAAAAAA | |
| DNASIS | |
| SEQ | |
| MRLLSVTFWL LLSLRTVVCV MEVQWCTISD AEQQKCKDMS EAFQGAGIRP SLLCVQGNSA | SEQ.ID.NO.:2 |
| DHCVQLIKEQ KADAITLDGG AIYEAGKEHG LKPVVGEVYD QDIGTSYYAV AVVRRNSNVT | |
| TNTLKGVKSC HTGINRTVGW NVFVGYLVES GHLSVMGCDV LKAVGDYFGG SCVPGTGETS | |
| HSESLCRLCR GDSSGRNVCD KSPLERYYDY SGAFRCLAEG AGDVAFVKHS TVLENTDGNT | |
| LPSWGKSLMS EDFQLLCRDG SRADITEWRR CHLAKVPAHA VVVRGDMDGG LIFQLLNEGQ | |
| LLFSHEDSSF QMFSSKAYSQ KNLLFKDSTL ELVPIATQNY EAWLGQEYLQ AMKGLLCDPN | |
| RLPHYLRWCV LSAPEIQKCG DMAVAFSRQN LKPEIQCVSA ESPEHCMEQI QAGHTDAVTL | |
| RGEDIYRAGK VYGLVPAAGE LYAEEDRSNS YFVVAVARRD SSYSFTLDEL RGKRSCHPYL | |
| GSPAGWEVPI GSLIQRGFIR PKDCDVLTAV SQFFNASCVP VNNPKNYPSA LCALCVGDEK | |
| GRNKCVGSSQ ERYYGYSGAF RCLVEHAGDV AFVKHTTVFE NTNGHNPEPW ASHLRWQDYE | |
| LLCPNGARAE VDQFQACNLA QMPSHAVMVR PDTNIFTVYG LLDKAQDLFG DDHNKNGFQM | |
| FDSSKYHSQD LLFKDATVRA VPVREKTTYL DWLGPDYVVA LEGMLSQQCS GAGAAVERVP | |
| LLALLLLTLA AGLLPRVL | |

We claim:

1. A method for screening a therapeutic agent for treating Alzheimer's disease (AD) comprising administering the agent to a mouse having an elevated level of mp97, and measuring the level of mp97 wherein a decrease in levels of mp97 indicates that the agent may be useful in treating Alzheimer's disease.

2. A method according to claim 1 wherein the level of mp97 is measured in the serum of the mouse.

3. A method according to claim 2 wherein the level of mp97 is measured using a radioimmunoassay, competitive assay or enzyme linked immunosorbant assay.

4. A method for assessing the ability of an agent to cross the blood brain barrier comprising (1) administering an effective amount of (a) the agent associated with murine p97 or (b) the agent associated with a compound that binds murine p97 and (2) testing the levels of the agent in the nervous system.

5. A method to assess the ability of a therapeutic agent to treat a neurological condition, comprising (1) administering to a mouse an effective amount of (a) the agent associated with murine p97 or (b) the agent associated with a compound that binds murine p97 and (2) monitoring the result of administration wherein an improvement in the neurological condition indicates that the agent has therapeutic effect.

6. A method according to claim 5 wherein the neurological condition is selected from the group consisting of cancers, neurodegenerative diseases, demyelinating diseases, amyotrophic lateral sclerosis, bacterial and viral infections, deficiency diseases, epilepsy, psychosis, pain and neurological disorders.

7. A method according to claim 5 wherein the neurological condition is Alzheimer's disease.

8. A method for identifying a compound that affects murine p97 protein activity or expression comprising:
(a) incubating a test compound with a murine p97 protein or a nucleic acid encoding a murine p97 protein; and
(b) determining an amount of murine p97 protein activity or expression and comparing with a control wherein a change in the murine p97 protein activity or expression as compared to the control indicates that the test compound has an effect on murine p97 protein activity or expression.

9. A method according to claim 8 wherein the p97 is expressed in a cell.

10. A method according to claim 9 wherein the cell expresses p97 as a fusion protein with a reporter gene.

11. A method of identifying substances which can bind with murine p97, comprising the steps of:
(a) reacting murine p97 and a test substance, under conditions which allow for formation of a complex between the murine p97 and the test substance, and
(b) assaying for complexes of murine p97 and the test substance, for free substance or for non complexed murine p97, wherein the presence of complexes indicates that the test substance is capable of binding murine p97.

12. A method according to any one of claims 1 to 11 wherein the p97 has an amino acid sequence shown in SEQ.ID.NO.: 2.

13. A transgenic non-human animal having increased expression of p97.

14. A transgenic non-human animal having decreased expression of p97.

15. A transgenic animal according to claim 13 or 14 wherein said animal is a mouse.

16. A use of a transgenic naimal according to any one of claims 13 to 15 to test therapeutic agents.

17. A use according to claim 16 to test therapeutic agents to treat neurological conditions.

18. A use according to claim 17 to test therapeutic agents to treat Alzheimer's disease.

19. A substantially isolated mp97 protein having at least 80% sequence identity with the amino acid sequence of SEQ ID NO: 2.

20. The substantially isolated mp97 protein of claim 19 wherein the sequence identity is at least 90%.

21. The substantially isolated mp97 protein of claim 19 wherein the sequence identity is 100%.

22. A substantially isolated mp97 protein having at least 80% sequence identity with amino acids 1 to 718 of SEQ ID NO: 2.

23. The substantially isolated mp97 protein of claim 22, wherein the sequence identity is at least 90%.

24. The substantially isolated mp97 protein of claim 22, wherein the sequence identity is 100%.

25. A substantially isolated nucleic acid sequence encoding a mp97 protein wherein the mp97 protein has at least 80% sequence identity with SEQ ID NO: 1.

26. A substantially isolated nucleic acid sequence encoding a mp97 protein wherein the mp97 protein has at least 90% sequence identity with SEQ ID NO: 1.

27. A substantially isolated nucleic acid sequence encoding a mp97 protein wherein the mp97 protein has at least 80% sequence identity with SEQ ID NO: 1.

28. An antibody that binds to a protein according to any one of claims 19 to 24.

29. An antisense oligonucleotide that is complimentary to a nucleic acid sequence according to claim 25 to 28.


SEQUENCE LISTING (103 sequences)

SEQ ID NO: 1 - DNA, 4068 bp, Mus musculus
  mouse p97, melanotransferrin, melanoma tumor-associated antigen
tggcctactgggaagaggaagccaggacagacccgccagcaccccagccaacccaacgttgccatgaggctcctgagcgtgactttttggctactcctgtccctgcgcactgtcgtctgtgtgatggaggtgcagtggtgtaccatctcagacgcagagcagcagaagtgcaaagacatgagcgaggccttccagggagctggcattcgtccttcccttctctgcgtccagggcaactccgctgaccactgtgtccagctcatcaaggaacaaaaagcagatgccatcaccctggatggaggggccatctatgaggcagggaaggagcacggcctgaagccagtggtgggggaagtctatgaccaagacattgggacttcctattatgccgtggctgtggtcaggaggaattccaatgttaccatcaacaccctgaagggcgtcaagtcctgccacacaggcattaaccggactgtgggctggaacgtgcctgtcggttacctcgtagagagcggccatctgtcagtgatgggctgtgatgtgctcaaagccgttggtgattattttggaggcagctgtgtccctggaacaggagaaaccagccattccgagtccctctgtcgcctctgccgtggcgactcttctgggcacaatgtgtgtgacaagagtcccctagagagatactacgactacagtggagccttccggtgcctggcggaaggagccggtgacgtggccttcgtgaagcacagcacagtgctggaaaatactgatggaaacaccctgccttcctggggcaagtccctgatgtcagaggacttccagctactatgcagggatggcagccgagccgacatcactgagtggagacgttgccacctggccaaggtgcctgctcatgctgtggtggtcaggggtgacatggatggcggtctcatattccaactgctcaacgaaggccagcttctgttcagccaygaagacagcagcttccagatgttcagctccaaagcctacagccagaagaacttgctgttcaaagactccaccttggagcttgtgcccattgccacacagaactatgaggcctggctgggccaggaatacctgcaggccatgaaggggctcctctgtgatcccaaccggctgccccactacctgcgctggtgtgtgctgtcagcgcccgagatccagaagtgtggagatatggctgtggccttcagccgccagaatctcaagccggaaattcagtgtgtgtcggccgagtcccctgagcactgcatggagcagatccaggctgggcacactgacgctgtgactctgaggggcgaggacatttacagggcaggaaaggtgtacggcctggttccggcggccggggagctgtatgctgaggaggacaggagcaattcctactttgtggtggctgtggcaagaagggacagctcctactccttcaccctggacgagcttcgcggcaagcgttcctgccacccctacttgggcagcccagcgggctgggaggtgcccatcggctccctcatccagcggggcttcatccggcccaaggactgtgatgtcctcacagcggtgagccagttcttcaatgccagctgcgtgcctgtcaacaaccctaagaactacccttccgcactatgtgcgctctgcgtgggagacgagaagggccgcaacaaatgtgtggggagcagccaggagagatactacggctacagcggggccttcaggtgccttgtggagcatgcaggggacgtggctttcgtcaagcacacgactgtctttgagaacacaaatggtcacaatcctgagccttgggcttctcacctcaggtggcaagactatgaactactgtgccccaatggggcacgggctgaggtagaccagttccaagcttgcaacctggcacaaatgccatcccacgctgtcatggtccgtccagacaccaacatcttcactgtgtatggacttctggacaaggcccaggacctgtttggagacgaccataacaagaacggtttccaaatgtttgactcctccaaatatcacagccaagacctgcttttcaaagatgctacagtccgagcggtgccagtccgggagaaaaccacatacctggactggctgggtcctgactatgtggttgcgctggaggggatgttgtctcagcagtgctccggtgcaggggccgcggtcgagcgagtccccctgctggccctgctcctgctgaccctggctgcaggcctccttcctcgcgttctctgaagaccgctgcttcaggccacgcccagagcagggaaagctacagagctcaaccggaagaaaccaggacatcagctaaccctgcaggagagcgcggggcgggatgaggagaggcaaggtgagaactcacacacacacacaagcctccgaggtgcgattctaacccaaagagaaatttctagaatcaggatgattgttaaggccaagtcttcccacttgctggagccctcaatacctgaggcgactggcgagtacgccagtcactcctcccacaccggtggcgccagcagcgaacctgtgcctcccacctggagcctcctggctggctggggtggttaagggggggggggggagagtgaagatgctggttgccatggcaaccgtggagcagcttccagcctctgtaccggccacctggtgagatgccaaggaaggagcacaccaccaacctagggaacctgtgcgacacactaccacccagcagcccctgctttcgctgccccaccgctctttcctatgggcacttgtccaccaaggccacaccgtcggaggggcaaggctgctgagcacatcagccttctgatgtgacaccaaccaaggagcccagccctctggacagcaagttttgctagactgggatgggaggaaggccagagctgtactgtggggatgaagtcctccaaaacctcagaggaaggaagtgcccccaccttcccattaagaatgttagtgtgtgagaaacttgatgcagggtggaaactatcctgtttaacggctcccgtggcaagcaggacttgcgctgtctgcgctgcctggacctcactgcacaatgaaactgttgccgagattctattgtttgctctcctggtctcagtctcaacattagttttctccctgccttcatataccccttcccacatcaccacgcaagcacgcacgcgcacacgcacacgcacacaccttatccgtgtgaacatatctgaacatatctgcttgtctgaagaagtaggagctaacccaaaataacttcctgtcatgagctgggccttgggatataccacgagccaggggattggggagagccctgtcttcccttcaccctgcacctgttgggcagttgcatctttcgagaggatccctggttctctcgaactgtgagagccaaggcctaggctgccattttgccattgttctctcgagaaccagaaaaagttttccaaagctaccagctcttaccccagatcttgttcccttaaaaaaaagtaataaataaaaaggagaagaaacaggagcaaacagccatcgtcagcacactggaagcagcgtgggccgggagctatttgtgtcttggtctgtgtggggggcctcagatcccaatgacaggccaggttcccagtggctcgcccccacctgtgggcgacgacgggacagatcctttccatggctcaccagtagagaaggtcctggcagtgtcccagccagagtcacacaatcctgaggaaaatcggtcaccatggtgcttgggagagcaagcccctcctcctcccagtacacagccatccattcttctctgagctggggacttcacagtgagaagtgtactctgtgtgggcgactgtgctgcccaaagtgtgatgtctgtgccgtgtgcctttcaggtgtgactttgaagagcgttgtgtaaatgacgtctgattgccatgggccactgctgtgtttgtgctaaagaaagacattggtttctttttaaaataaagccatatatccctgcaaaaaaaaaaaaaaaaaaaa

SEQ ID NO: 2 - protein, 738 aa, Mus musculus
  mouse p97, melanotransferrin, melanoma tumor-associated antigen
MRLLSVTFWLLLSLRTVVCVMEVQWCTISDAEQQKCKDMSEAFQGAGIRPSLLCVQGNSADHCVQLIKEQKADAITLDGGAIYEAGKEHGLKPVVGEVYDQDIGTSYYAVAVVRRNSNVTINTLKGVKSCHTGINRTVGWNVPVGYLVESGHLSVMGCDVLKAVGDYFGGSCVPGTGETSHSESLCRLCRGDSSGHNVCDKSPLERYYDYSGAFRCLAEGAGDVAFVKHSTVLENTDGNTLPSWGKSLMSEDFQLLCRDGSRADITEWRRCHLAKVPAHAVVVRGDMDGGLIFQLLNEGQLLFSHEDSSFQMFSSKAYSQKNLLFKDSTLELVPIATQNYEAWLGQEYLQAMKGLLCDPNRLPHYLRWCVLSAPEIQKCGDMAVAFSRQNLKPEIQCVSAESPEHCMEQIQAGHTDAVTLRGEDIYRAGKVYGLVPAAGELYAEEDRSNSYFVVAVARRDSSYSFTLDELRGKRSCHPYLGSPAGWEVPIGSLIQRGFIRPKDCDVLTAVSQFFNASCVPVNNPKNYPSALCALCVGDEKGRNKCVGSSQERYYGYSGAFRCLVEHAGDVAFVKHTTVFENTNGHNPEPWASHLRWQDYELLCPNGARAEVDQFQACNLAQMPSHAVMVRPDTNIFTVYGLLDKAQDLFGDDHNKNGFQMFDSSKYHSQDLLFKDATVRAVPVREKTTYLDWLGPDYVVALEGMLSQQCSGAGAAVERVPLLALLLLTLAAGLLPRVL

SEQ ID NO: 3 - protein, 738 aa, Homo sapiens
  human p97, melanotransferrin, melanoma tumor-associated antigen
MRGPSGALWLLLALRTVLGGMEVRWCATSDPEQHKCGNMSEAFREAGIQPSLLCVRGTSADHCVQLIAAQEADAITLDGGAIYEAGKEHGLKPVVGEVYDQEVGTSYYAVAVVRRSSHVTIDTLKGVKSCHTGINRTVGWNVPVGYLVESGRLSVMGCDVLKAVSDYFGGSCVPGAGETSYSESLCRLCRGDSSGEGVCDKSPLERYYDYSGAFRCLAEGAGDVAFVKHSTVLENTDGKTLPSWGQALLSQDFELLCRDGSRADVTEWRQCHLARVPAHAVVVRADTDGGLIFRLLNEGQRLFSHEGSSFQMFSSEAYGQKDLLFKDSTSELVPIATQTYEAWLGHEYLHAMKGLLCDPNRLPPYLRWCVLSTPEIQKCGDMAVAFRRQRLKPEIQCVSAKSPQHCMERIQAEQVDAVTLSGEDIYTAGKKYGLVPAAGEHYAPEDSSNSYYVVAVVRRDSSHAFTLDELRGKRSCHAGFGSPAGWDVPVGALIQRGFIRPKDCDVLTAVSEFFNASCVPVNNPKNYPSSLCALCVGDEQGRNKCVGNSQERYYGYRGAFRCLVENAGDVAFVRHTTVFDNTNGHNSEPWAAELRSEDYELLCPNGARAEVSQFAACNLAQIPPHAVMVRPDTNIFTVYGLLDKAQDLFGDDHNKNGFKMFDSSNYHGQDLLFKDATVRAVPVGEKTTYRGWLGLDYVAALEGMSSQQCSGAAAPAPGAPLLPLLLPALAARLLPPAL

SEQ ID NO: 4 - protein, 4 aa, Artificial Sequence
  Description of Artificial Sequencemp97 and hp97 homology consensus peptide
WLLL

SEQ ID NO: 5 - protein, 4 aa, Artificial Sequence
  Description of Artificial Sequencemp97 and hp97 homology consensus peptide
LRTV

SEQ ID NO: 6 - protein, 5 aa, Artificial Sequence
  Description of Artificial Sequencemp97 and hp97 homology consensus peptide
MSEAF

SEQ ID NO: 7 - protein, 6 aa, Artificial Sequence
  Description of Artificial Sequencemp97 and hp97 homology consensus peptide
PSLLCV

SEQ ID NO: 8 - protein, 9 aa, Artificial Sequence
  Description of Artificial Sequencemp97 and hp97 homology consensus peptide
SADHCVQLI

SEQ ID NO: 9 - protein, 30 aa, Artificial Sequence
  Description of Artificial Sequencemp97 and hp97 homology consensus peptide
ADAITLDGGAIYEAGKEHGLKPVVGEVYDQ

SEQ ID NO: 10 - protein, 12 aa, Artificial Sequence
  Description of Artificial Sequencemp97 and hp97 homology consensus peptide
GTSYYAVAVVRR

SEQ ID NO: 11 - protein, 29 aa, Artificial Sequence
  Description of Artificial Sequencemp97 and hp97 homology consensus peptide
TLKGVKSCHTGINRTVGWNVPVGYLVESG

SEQ ID NO: 12 - protein, 12 aa, Artificial Sequence
  Description of Artificial Sequencemp97 and hp97 homology consensus peptide
LSVMGCDVLKAV

SEQ ID NO: 13 - protein, 10 aa, Artificial Sequence
  Description of Artificial Sequencemp97 and hp97 homology consensus peptide
DYFGGSCVPG

SEQ ID NO: 14 - protein, 4 aa, Artificial Sequence
  Description of Artificial Sequencemp97 and hp97 homology consensus peptide
GETS

SEQ ID NO: 15 - protein, 14 aa, Artificial Sequence
  Description of Artificial Sequencemp97 and hp97 homology consensus peptide
SESLCRLCRGDSSG

SEQ ID NO: 16 - protein, 41 aa, Artificial Sequence
  Description of Artificial Sequencemp97 and hp97 homology consensus peptide
VCDKSPLERYYDYSGAFRCLAEGAGDVAFVKHSTVLENTDG

SEQ ID NO: 17 - protein, 6 aa, Artificial Sequence
  Description of Artificial Sequencemp97 and hp97 homology consensus peptide
TLPSWG

SEQ ID NO: 18 - protein, 10 aa, Artificial Sequence
  Description of Artificial Sequencemp97 and hp97 homology consensus peptide
LLCRDGSRAD

SEQ ID NO: 19 - protein, 4 aa, Artificial Sequence
  Description of Artificial Sequencemp97 and hp97 homology consensus peptide
TEWR

SEQ ID NO: 20 - protein, 4 aa, Artificial Sequence
  Description of Artificial Sequencemp97 and hp97 homology consensus peptide
CHLA

SEQ ID NO: 21 - protein, 9 aa, Artificial Sequence
  Description of Artificial Sequencemp97 and hp97 homology consensus peptide
VPAHAVVVR

SEQ ID NO: 22 - protein, 6 aa, Artificial Sequence
  Description of Artificial Sequencemp97 and hp97 homology consensus peptide
DGGLIF

SEQ ID NO: 23 - protein, 6 aa, Artificial Sequence
  Description of Artificial Sequencemp97 and hp97 homology consensus peptide
LLNEGQ

SEQ ID NO: 24 - protein, 5 aa, Artificial Sequence
  Description of Artificial Sequencemp97 and hp97 homology consensus peptide
LFSHE

SEQ ID NO: 25 - protein, 8 aa, Artificial Sequence
  Description of Artificial Sequencemp97 and hp97 homology consensus peptide
SSFQMFSS

SEQ ID NO: 26 - protein, 7 aa, Artificial Sequence
  Description of Artificial Sequencemp97 and hp97 homology consensus peptide
LLFKDST

SEQ ID NO: 27 - protein, 8 aa, Artificial Sequence
  Description of Artificial Sequencemp97 and hp97 homology consensus peptide
ELVPIATQ

SEQ ID NO: 28 - protein, 6 aa, Artificial Sequence
  Description of Artificial Sequencemp97 and hp97 homology consensus peptide
YEAWLG

SEQ ID NO: 29 - protein, 13 aa, Artificial Sequence
  Description of Artificial Sequencemp97 and hp97 homology consensus peptide
AMKGLLCDPNRLP

SEQ ID NO: 30 - protein, 8 aa, Artificial Sequence
  Description of Artificial Sequencemp97 and hp97 homology consensus peptide
YLRWCVLS

SEQ ID NO: 31 - protein, 13 aa, Artificial Sequence
  Description of Artificial Sequencemp97 and hp97 homology consensus peptide
PEIQKCGDMAVAF

SEQ ID NO: 32 - protein, 10 aa, Artificial Sequence
  Description of Artificial Sequencemp97 and hp97 homology consensus peptide
LKPEIQCVSA

SEQ ID NO: 33 - protein, 4 aa, Artificial Sequence
  Description of Artificial Sequencemp97 and hp97 homology consensus peptide
HCME

SEQ ID NO: 34 - protein, 5 aa, Artificial Sequence
  Description of Artificial Sequencemp97 and hp97 homology consensus peptide
DAVTL

SEQ ID NO: 35 - protein, 5 aa, Artificial Sequence
  Description of Artificial Sequencemp97 and hp97 homology consensus peptide
GEDIY

SEQ ID NO: 36 - protein, 9 aa, Artificial Sequence
  Description of Artificial Sequencemp97 and hp97 homology consensus peptide
YGLVPAAGE

SEQ ID NO: 37 - protein, 4 aa, Artificial Sequence
  Description of Artificial Sequencemp97 and hp97 homology consensus peptide
SNSY

SEQ ID NO: 38 - protein, 4 aa, Artificial Sequence
  Description of Artificial Sequencemp97 and hp97 homology consensus peptide
VVAV

SEQ ID NO: 39 - protein, 5 aa, Artificial Sequence
  Description of Artificial Sequencemp97 and hp97 homology consensus peptide
RRDSS

SEQ ID NO: 40 - protein, 13 aa, Artificial Sequence
  Description of Artificial Sequencemp97 and hp97 homology consensus peptide
FTLDELRGKRSCH

SEQ ID NO: 41 - protein, 6 aa, Artificial Sequence
  Description of Artificial Sequencemp97 and hp97 homology consensus peptide
GSPAGW

SEQ ID NO: 42 - protein, 19 aa, Artificial Sequence
  Description of Artificial Sequencemp97 and hp97 homology consensus peptide
LIQRGFIRPKDCDVLTAVS

SEQ ID NO: 43 - protein, 17 aa, Artificial Sequence
  Description of Artificial Sequencemp97 and hp97 homology consensus peptide
FFNASCVPVNNPKNYPS

SEQ ID NO: 44 - protein, 9 aa, Artificial Sequence
  Description of Artificial Sequencemp97 and hp97 homology consensus peptide
LCALCVGDE

SEQ ID NO: 45 - protein, 7 aa, Artificial Sequence
  Description of Artificial Sequencemp97 and hp97 homology consensus peptide
GRNKCVG

SEQ ID NO: 46 - protein, 8 aa, Artificial Sequence
  Description of Artificial Sequencemp97 and hp97 homology consensus peptide
SQERYYGY

SEQ ID NO: 47 - protein, 8 aa, Artificial Sequence
  Description of Artificial Sequencemp97 and hp97 homology consensus peptide
GAFRCLVE

SEQ ID NO: 48 - protein, 7 aa, Artificial Sequence
  Description of Artificial Sequencemp97 and hp97 homology consensus peptide
AGDVAFV

SEQ ID NO: 49 - protein, 5 aa, Artificial Sequence
  Description of Artificial Sequencemp97 and hp97 homology consensus peptide
HTTVF

SEQ ID NO: 50 - protein, 6 aa, Artificial Sequence
  Description of Artificial Sequencemp97 and hp97 homology consensus peptide
NTNGHN

SEQ ID NO: 51 - protein, 4 aa, Artificial Sequence
  Description of Artificial Sequencemp97 and hp97 homology consensus peptide
EPWA

SEQ ID NO: 52 - protein, 14 aa, Artificial Sequence
  Description of Artificial Sequencemp97 and hp97 homology consensus peptide
DYELLCPNGARAEV

SEQ ID NO: 53 - protein, 6 aa, Artificial Sequence
  Description of Artificial Sequencemp97 and hp97 homology consensus peptide
ACNLAQ

SEQ ID NO: 54 - protein, 34 aa, Artificial Sequence
  Description of Artificial Sequencemp97 and hp97 homology consensus peptide
HAVMVRPDTNIFTVYGLLDKAQDLFGDDHNKNGF

SEQ ID NO: 55 - protein, 5 aa, Artificial Sequence
  Description of Artificial Sequencemp97 and hp97 homology consensus peptide
MFDSS

SEQ ID NO: 56 - protein, 15 aa, Artificial Sequence
  Description of Artificial Sequencemp97 and hp97 homology consensus peptide
QDLLFKDATVRAVPV

SEQ ID NO: 57 - protein, 5 aa, Artificial Sequence
  Description of Artificial Sequencemp97 and hp97 homology consensus peptide
EKTTY

SEQ ID NO: 58 - protein, 5 aa, Artificial Sequence
  Description of Artificial Sequencemp97 and hp97 homology consensus peptide
ALEGM

SEQ ID NO: 59 - protein, 7 aa, Artificial Sequence
  Description of Artificial Sequencemp97 and hp97 homology consensus peptide
SQQCSGA

SEQ ID NO: 60 - protein, 19 aa, Artificial Sequence
  Description of Artificial Sequencemurine p97 N-terminal signal peptide
MRLLSVTFWLLLSLRTVVC

SEQ ID NO: 61 - protein, 5 aa, Artificial Sequence
  Description of Artificial Sequencemurine p97 signal peptide cleavage position
VVCVM

SEQ ID NO: 62 - protein, 20 aa, Artificial Sequence
  Description of Artificial Sequencemurine p97 hydrophobic tail
VPLLALLLLTLAAGLLPRVL

SEQ ID NO: 63 - protein, 4 aa, Artificial Sequence
  Description of Artificial Sequencemurine p97 N-glycosylation site
NVTI

SEQ ID NO: 64 - protein, 4 aa, Artificial Sequence
  Description of Artificial Sequencemurine p97 N-glycosylation site
NRTV

SEQ ID NO: 65 - protein, 4 aa, Artificial Sequence
  Description of Artificial Sequencemurine p97 N-glycosylation site
NASC

SEQ ID NO: 66 - protein, 10 aa, Artificial Sequence
  Description of Artificial Sequencemurine p97 transferrin iron binding Motif I N-Lobe
YYAVAVVRRN

SEQ ID NO: 67 - protein, 10 aa, Artificial Sequence
  Description of Artificial Sequencemurine p97 transferrin iron binding Motif I C-Lobe
YFVVAVARRD

SEQ ID NO: 68 - protein, 17 aa, Artificial Sequence
  Description of Artificial Sequencemurine p97 transferrin iron binding Motif II N-Lobe
YSGAFRCLAEGAGDVAF

SEQ ID NO: 69 - protein, 17 aa, Artificial Sequence
  Description of Artificial Sequencemurine p97 transferrin iron binding Motif II C-Lobe
YSGAFRCLVEHAGDVAF

SEQ ID NO: 70 - protein, 31 aa, Artificial Sequence
  Description of Artificial Sequencemurine p97 transferrin iron binding Motif III N-Lobe
DFQLLCRDGSRADITEWRRCHLAKVPAHAVV

SEQ ID NO: 71 - protein, 31 aa, Artificial Sequence
  Description of Artificial Sequencemurine p97 transferrin iron binding Motif III C-Lobe
DYELLCPNGARAEVDQFQACNLAQMPSHAVM

SEQ ID NO: 72 - protein, 8 aa, Artificial Sequence
  Description of Artificial Sequencemurine p97 tyrosine kinase phosphorylation site
KSPLERYY

SEQ ID NO: 73 - protein, 9 aa, Artificial Sequence
  Description of Artificial Sequencemurine p97 Myc-type helix-loop-helix dimerization motif
STLELVPIA

SEQ ID NO: 74 - protein, 7 aa, Artificial Sequence
  Description of Artificial Sequencemurine p97 immunoglobulin and major histocompatibility complex protein motif
FRCLVEH

SEQ ID NO: 75 - protein, 4 aa, Artificial Sequence
  Description of Artificial Sequencemurine p97 glycosaminoglycan attachment site
SGAG

SEQ ID NO: 76 - DNA, 28 bp, Artificial Sequence
  Description of Artificial Sequencemutagenic primer
ggggccgcggtcgagtgagtccccctgg

SEQ ID NO: 77 - DNA, 40 bp, Artificial Sequence
  Description of Artificial Sequenceselection primer
cattttgccattgttctcccgggaaccagaaaaagttttc

SEQ ID NO: 78 - protein, 6 aa, Artificial Sequence
  Description of Artificial SequenceHis6-tag
HHHHHH

SEQ ID NO: 79 - DNA, 33 bp, Artificial Sequence
  Description of Artificial SequenceHis6-tag complementary synthetic oligo
ggtcgagcgacatcatcatcatcatcattgagc

SEQ ID NO: 80 - DNA, 33 bp, Artificial Sequence
  Description of Artificial SequenceHis6-tag complementary synthetic oligo
tcaatgatgatgatgatgatgtcgctcgaccgc

SEQ ID NO: 81 - DNA, 29 bp, Artificial Sequence
  Description of Artificial SequencemMTf+1 oligonucleotide used in cloning mp97
gactcaagcttgccagctgcgtgcctgtc

SEQ ID NO: 82 - DNA, 19 bp, Artificial Sequence
  Description of Artificial SequencemMTf+2 oligonucleotide used in cloning mp97
gtggtggctgtggctagaa

SEQ ID NO: 83 - DNA, 19 bp, Artificial Sequence
  Description of Artificial SequencemMTf+3 oligonucleotide used in cloning mp97
ttcccaacatcaccaacgc

SEQ ID NO: 84 - DNA, 21 bp, Artificial Sequence
  Description of Artificial SequencemMTf+4 oligonucleotide used in cloning mp97
ctggacaaggcccaggacctg

SEQ ID NO: 85 - DNA, 18 bp, Artificial Sequence
  Description of Artificial SequencemMTf+5 oligonucleotide used in cloning mp97
tgagggagaggcaaggtg

SEQ ID NO: 86 - DNA, 18 bp, Artificial Sequence
  Description of Artificial SequencemMTf+6 oligonucleotide used in cloning mp97
gccagagctgtactgtgg

SEQ ID NO: 87 - DNA, 22 bp, Artificial Sequence
  Description of Artificial SequencemMTf+7 oligonucleotide used in cloning mp97
cttatccgtgtgaacatatctg

SEQ ID NO: 88 - DNA, 18 bp, Artificial Sequence
  Description of Artificial SequencemMTf+8 oligonucleotide used in cloning mp97
tggagacgttgccacctg

SEQ ID NO: 89 - DNA, 18 bp, Artificial Sequence
  Description of Artificial SequencemMTf+9 oligonucleotide used in cloning mp97
tctgtcgcctctgccgtg

SEQ ID NO: 90 - DNA, 31 bp, Artificial Sequence
  Description of Artificial SequencemMTf-1 oligonucleotide used in cloning mp97
gtcaaggatccgaaggccacagccatatctc

SEQ ID NO: 91 - DNA, 19 bp, Artificial Sequence
  Description of Artificial SequencemMTf-2 oligonucleotide used in cloning mp97
gcgttggtgatgttgggaa

SEQ ID NO: 92 - DNA, 19 bp, Artificial Sequence
  Description of Artificial SequencemMTf-3 oligonucleotide used in cloning mp97
ttctagccacagccaccac

SEQ ID NO: 93 - DNA, 24 bp, Artificial Sequence
  Description of Artificial SequencemMTf-4 oligonucleotide used in cloning mp97
gctcctacttcttcagacaagcag

SEQ ID NO: 94 - DNA, 26 bp, Artificial Sequence
  Description of Artificial SequencemMTf-5 oligonucleotide used in cloning mp97
tgcatgctccacaaggcacctgaagg

SEQ ID NO: 95 - DNA, 21 bp, Artificial Sequence
  Description of Artificial SequencemMTf-6 oligonucleotide used in cloning mp97
caggtcctgggccttgtccag

SEQ ID NO: 96 - DNA, 18 bp, Artificial Sequence
  Description of Artificial SequencemMTf-7 oligonucleotide used in cloning mp97
ccacagtacagctctggc

SEQ ID NO: 97 - DNA, 18 bp, Artificial Sequence
  Description of Artificial SequencemMTf-8 oligonucleotide used in cloning mp97
caccttgcctctccctca

SEQ ID NO: 98 - DNA, 19 bp, Artificial Sequence
  Description of Artificial SequencemMTf-9 oligonucleotide used in cloning mp97
aggcacaggttcgctgctg

SEQ ID NO: 99 - DNA, 18 bp, Artificial Sequence
  Description of Artificial SequencemMTf-10 oligonucleotide used in cloning mp97
agcagcggtcttcagaga

SEQ ID NO: 100 - DNA, 18 bp, Artificial Sequence
  Description of Artificial SequencemMTf-11 oligonucleotide used in cloning mp97
gctggaagtcctctgaca

SEQ ID NO: 101 - DNA, 30 bp, Artificial Sequence
  Description of Artificial SequencemMTf-12 oligonucleotide used in cloning mp97
gtgctagctagcgctctgcgtctgagatgg

SEQ ID NO: 102 - DNA, 20 bp, Artificial Sequence
  Description of Artificial SequencepME18S 5′ oligonucleotide used in cloning mp97
cttctgctctaaaagctgcg

SEQ ID NO: 103 - DNA, 20 bp, Artificial Sequence
  Description of Artificial SequencepME18S 3′ oligonucleotide used in cloning mp97
cgacctgcagctcgagcaca